(19) United States
(12) Patent Application Publication
(10) Pub. No.: US 2008/0207677 A1
(43) Pub. Date: Aug. 28, 2008

(54) Napthyridine Compounds As Rock Inhibitors

(75) Inventors: Stefan Muller, Munchen (DE); Wilfried Schwab, Velbert (DE); Bert Klebl, Oberschweinbach (DE); Doris Hafenbradl, Pullach (DE); Edmund Hoppe, Krailling (DE); Zoltan Horvath, Budapest (HU); Gyorgy Keri, Budapest (HU); Zoltan Varga, Kolesd (HU); Laszlo Orfi, Budapest (HU); Jeno Marosfalvi, Budapest (HU); Gerhard Muller, Utting (DE)

Correspondence Address: BROOKS KUSHMAN P.C., 1000 TOWN CENTER, TWENTY-SECOND FLOOR, SOUTHFIELD, MI, 48075, US

(73) Assignee: GPC BIOTECH AG, Planegg-Martinsried (DE)

(21) Appl. No.: 11/813,236

(22) PCT Filed: Jan. 2, 2006
(86) PCT No.: PCT/EP06/50005

§ 371 Date: Sep. 26, 2007

(30) Foreign Application Priority Data
Dec. 31, 2004 (DE) 04031078.1

(51) Int. Cl. A61K 31/4375; C07D 471/04; A61P 35/00

(52) U.S. Cl. 514/300; 546/122

(57) ABSTRACT

The present invention relates to compounds having a naphthyridine scaffold, and stereoisomeric forms, prodrugs, solvates, hydrates and/or pharmaceutically acceptable salts of these compounds as well as pharmaceutical compositions containing at least one of these naphthyridine derivatives together with pharmaceutically acceptable carrier, excipient and/or diluents. Said naphthyridine compounds have been identified as inhibitors of the protein kinase ROCK2, also known as Rho-kinase, and are useful for the treatment of cancers (tumor growth and metastases), erectile dysfunction, cardiovascular diseases, hypertension, angina pectoris, cerebral ischaemia, cerebral vasospasm, myocardial ischaemia, coronary vasospasm, heart failure, myocardial hypertrophy, atherosclerosis, restenosis, spinal cord injuries, neuronal degeneration, thrombotic disorders, asthma, glaucoma, inflammation, anti-viral diseases (e.g. HIV), and osteoporosis.

The present invention relates to compounds having a naphthyridine scaffold, and stereoisomeric forms, prodrugs, solvates, hydrates and/or pharmaceutically acceptable salts of these compounds as well as pharmaceutical compositions containing at least one of these naphthyridine derivatives together with pharmaceutically acceptable carrier, excipient and/or diluents. Said naphthyridine compounds have been identified as inhibitors of the ROCK protein kinases ROCK1 and ROCK2, also known as Rho-kinases, and are useful for the treatment of cancers (tumor growth and metastases), erectile dysfunction, cardiovascular diseases, hypertension, angina pectoris, cerebral ischaemia, cerebral vasospasm, myocardial ischaemia, coronary vasospasm, heart failure, myocardial hypertrophy, atherosclerosis, restenosis, spinal cord injuries, neuronal degeneration, thrombotic disorders, asthma, glaucoma, inflammation, anti-viral diseases (e.g. HIV), and osteoporosis.

### BACKGROUND OF THE INVENTION

WO 97/34894 A1 discloses naphthyridine compounds for the treatment of cytomegalovirus (CMV) infections. WO 99/29318 A1 describes the use of naphthyridine compounds for the inhibition of the replication of HIV, HBV, HCV, HSV-1, HSV-2, Parainfluenza, Influenza A, Influenza B, Adenovirus, RV and RVS.

It is object of the present invention to provide compounds, stereoisomeric forms and/or pharmaceutically acceptable salts thereof which can be used as pharmaceutically active agents, especially for prophylaxis and/or treatment of cancers (tumor growth and metastases), erectile dysfunction, cardiovascular diseases, hypertension, angina pectoris, cerebral ischaemia, cerebral vasospasm, myocardial ischaemia, coronary vasospasm, heart failure, myocardial hypertrophy, atherosclerosis, restenosis, spinal cord injuries, neuronal degeneration, thrombotic disorders, asthma, glaucoma, inflammation, anti-viral diseases (e.g. HIV), and osteoporosis.

The object of the present invention is solved by the teaching of the independent claims. Further advantageous features, aspects and details of the invention are evident from the dependent claims, the description, and the examples of the present application.

The novel naphthyridine derivatives according to the present invention are represented by the following general formula (I)

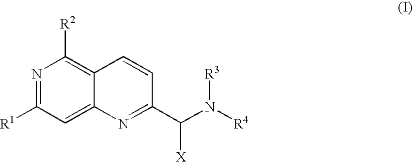

wherein
R¹, R², R²⁶ R⁶⁹ represent independently of each other H, OH, OCH₃, OC₂H₅, OC₃H₇, O cyclo-C₃H₅, OCH(CH₃)₂, OC(CH₃)₃, OC₄H₉, —OPh, —OCH₂-Ph, —OCPh₃, SH, SCH₃, SC₂H₅, SC₃H₇, S cyclo-C₃H₅, SCH(CH₃)₂, SC(CH₃)₃, NO₂, F, Cl, Br, I, N [symbol] , CN, OCN, NCO, SCN, NCS, CHO, COCH [symbol] , COC [symbol] H₅, COC [symbol] H₇, CO cyclo-C [symbol] H₅, COCH(CH [symbol] )₂, COC(CH [symbol] )₃, COOH, COCN, COOCH [symbol] , COOC [symbol] H₅, COOC [symbol] H₇, COO cyclo-C [symbol] H₅, COOCH(CH [symbol] )₂, COOC(CH [symbol] )₃, OOC CH(CH [symbol] )₂, OOC C [symbol] H₅, OOC C [symbol] H₇, OOC cyclo-C [symbol] H₅, OOC CH(CH [symbol] )₂, OOC C(CH [symbol] )₃, CONH [symbol] , CONHCH [symbol] , CONHC [symbol] H₅, CONHC [symbol] H₇, CONH cyclo-C [symbol] H₅, CONH[CH(CH [symbol] )₂], CONH[C(CH [symbol] )₃], CON(CH [symbol] )₂, CON(C [symbol] H₅)₂, CON(C [symbol] H₇)₂, CON(cyclo-C [symbol] H₅)₂, CON[CH(CH [symbol] )₂]₂, CON[C(CH [symbol] )₃]₂, NH₂, NHCH [symbol] , NHC [symbol] H₅, NHC [symbol] H₇, NH cyclo-C [symbol] H₅, NHCH(CH [symbol] )₂, NHC(CH [symbol] )₃, N(CH₃)₂, N(C [symbol] H₅)₂, N(C₆H₇)₂, N(cyclo-C [symbol] H₅)₂, N[CH(CH₃)₂]₂, N[C(CH [symbol] )₃]₂, SOCH [symbol] , SOC [symbol] H₅, SOC [symbol] H₇, SO cyclo-C [symbol] H₅, SOCH(CH [symbol] )₂, SOC(CH [symbol] )₃, SO [symbol] CH₃, SO [symbol] C₂H₅, SO₂C₃H₇, SO [symbol] cyclo-C [symbol] H₅, SO [symbol] CH(CH₃)₂, SO [symbol] C(CH₃)₃, SO [symbol] H, SO [symbol] CH₃, SO₃C₂H₅, SO [symbol] C₃H₇, SO [symbol] cyclo-C [symbol] H₅, SO [symbol] CH(CH₃)₂, SO [symbol] C(CH₃)₃, OCF [symbol] , OC₂F₅, O COOCH [symbol] , O COOC [symbol] H₅, O COOC [symbol] H₇, O COO cyclo-C [symbol] H₅, O COOCH(CH [symbol] )₂, O COOC(CH [symbol] )₃, NH CO NH [symbol] NH CO NHCH [symbol] , NH CO NHC [symbol] H₅, NH CO NHC [symbol] H₇, NH CO NH cyclo-C [symbol] H₅, NH CO NH[CH(CH [symbol] ) [symbol] ], NH CO NH[C(CH [symbol] )₃], NH CO N(CH [symbol] )₂, NH CO N(C [symbol] H₅)₂, NH CO N(C [symbol] H₇)₂, NH CO N(cyclo-C [symbol] H₅)₂, NH CO N[CH(CH [symbol] )₂]₂, NH CO N[C(CH [symbol] )₃]₂, NH CS NH [symbol] , NH CS NH cyclo-C [symbol] H [symbol] , NH CS NHC [symbol] H₇, NH CS NH[CH(Ch [symbol] )₂], NH CS NH[C(CH [symbol] )₃], NH CS N(CH [symbol] )₂, NH CS N(CH [symbol] )₂, NH CS N(C [symbol] H₇)₂, NH CS N(cyclo-C [symbol] H₅)₂, NH C(═NH) NH [symbol] , NH CS NHC [symbol] H₅, NH CS NHCH [symbol] , O CO NH [symbol] [symbol] . O CO NHCH [symbol] , O CO NH [symbol] H₅, O CO NHC [symbol] H₇, O CO NH cyclo-C [symbol] H₅, O CO NH[CH(CH [symbol] )₂], O CO NH[C(CH [symbol] )₃], O CO N(CH [symbol] )₂, O CO N(C [symbol] H₅)₂, O CO N(C [symbol] H₇)₂, O CO N(cyclo-C [symbol] H₅)₂, O CO N[CH(CH(CH [symbol] )₂]₂, O CO N[C(CH [symbol] )₃]₂, O CO OCH [symbol] , O CO OC [symbol] H₅, O CO OC [symbol] H₇, O CO O cyclo-C [symbol] H₅, O CO OCH(CH [symbol] )₂, O CO OC(CH [symbol] )₃, —CH₂F —CHF₂, —CF₃, —CH₂Cl, —CHCl₂, —CCl₃, —CH₂Br—CHBr₂, —CBr₃, —CPh₃, —CH₂—CH₂F—CH₂—CHF₂, —CH₂—CF₃, —CH₂—CH₂Cl, —CH₂—CHCl₂, —CH₂—CCl₃, —CH₂—CH₂Br—CH₂—CHBr₂, —CH₂—CBr₃, —CH₃, —C₂H₅, —C₃H₇, cyclo-C [symbol] H₅, —CH(CH₃)₂, —C(CH₃)₃, —C₄H₉, —CH₂—CH(CH₃)₂, —CH(CH₃)—C₂H₅, —C(CH₃)₃, —C₅H₁₁, —C₆H₁₃, —C₇H₁₅, —C₈H₁₇, —C₉H₁₉, —C₁₀H₂₁, -Ph, —CH₂-Ph, —CH═CH₂, —CH₂—CH═CH₂, —C(CH₃)═CH₂, —CH═CH—CH₃, C [symbol] H₄ CH═CH [symbol] , CH═C(CH [symbol] )₂, —C≡CH, —C≡C—CH₃, —CH₂—C≡CH;
R³, R¹¹ represent independently of each other —H, —CH₃, —C₂H₅, —C₃H₇, cyclo-C [symbol] H₅, —CH(CH₃)₂, —C(CH₃)₃, —C₄H₉, —CH₂—CH(CH₃)₂, —CH(CH₃)—C₂H₅, —C(CH₃)₃, —C₅H₁₁, —C₆H₁₃, —C₇H₁₅, —C₈H₁₇, —C₉H₁₉, —C₁₀H₂₁, -Ph, —CH₂-Ph, —CH═CH₂, —CH₂—CH═CH₂, —C(CH₃)═CH₂, —CH═CH—CH₃, C [symbol] H₄ CH═CH [symbol] , CH═C(CH [symbol] )₂, —C≡CH, —C≡C—CH₃, —CH₂C≡CH;
the moiety

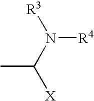

represents

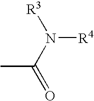

### or R*;

R⁴ represents R** or

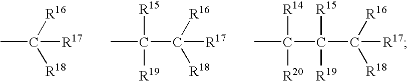

R* and R** represent independently of each other

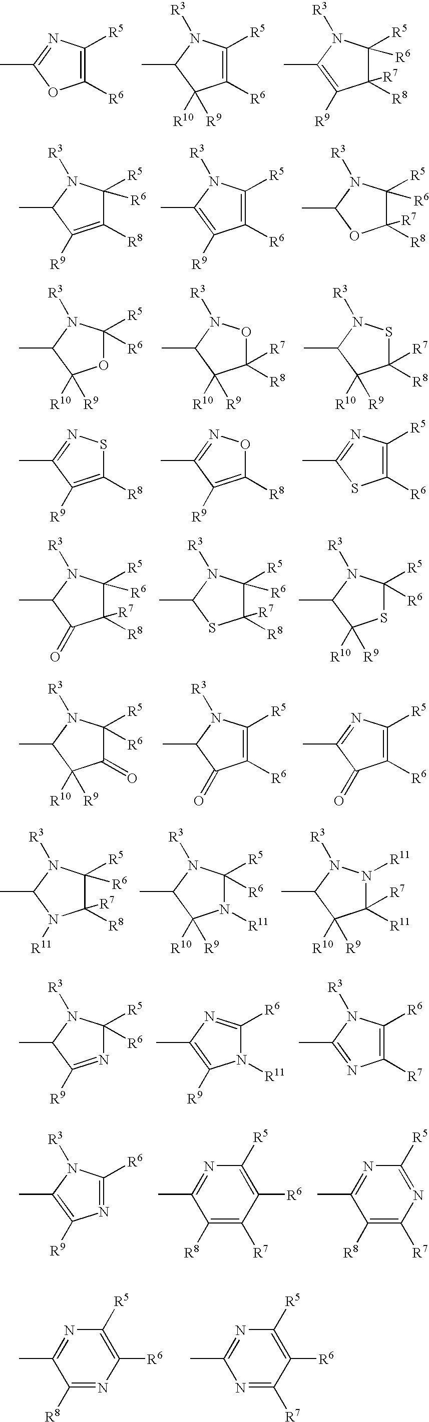

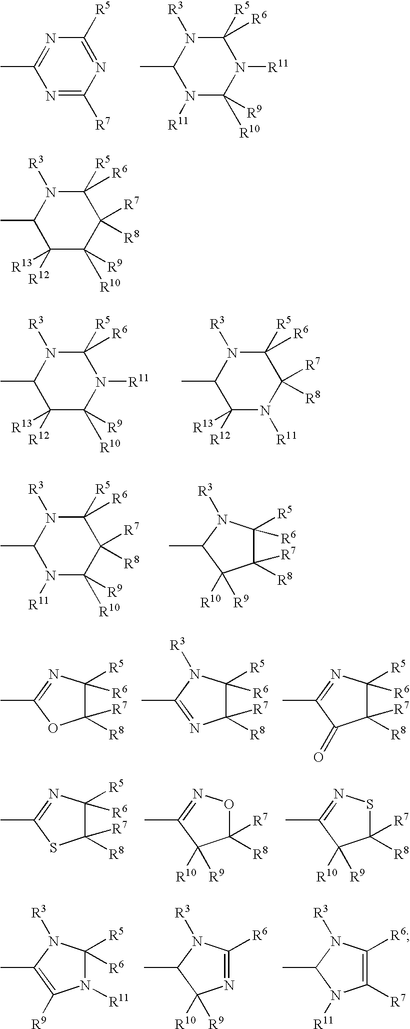

R# represents R** or

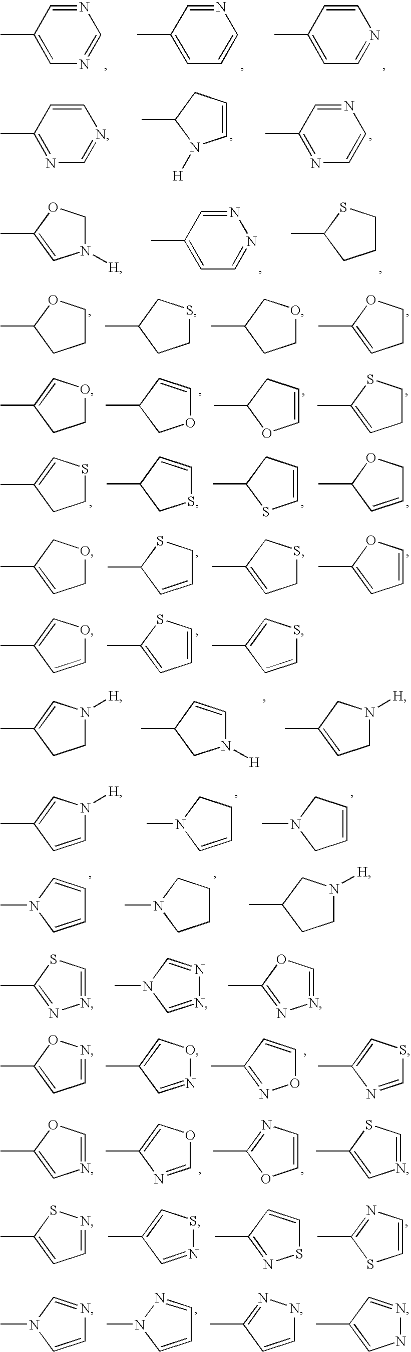

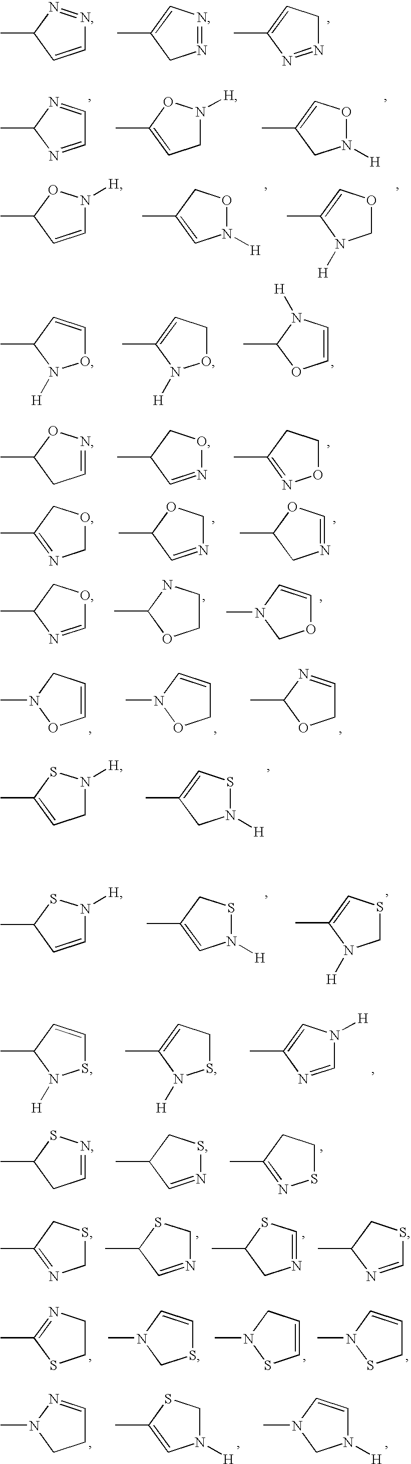

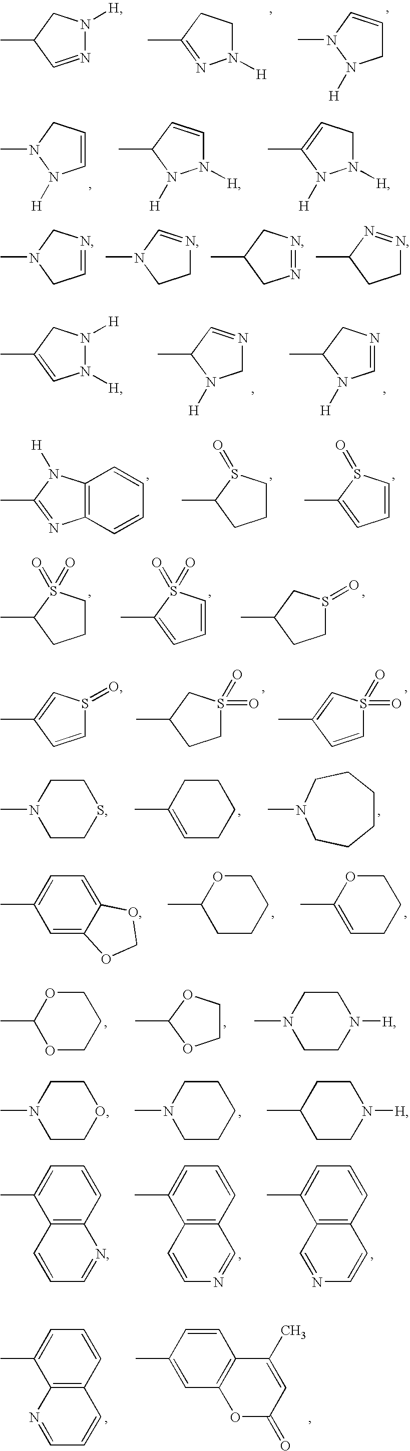

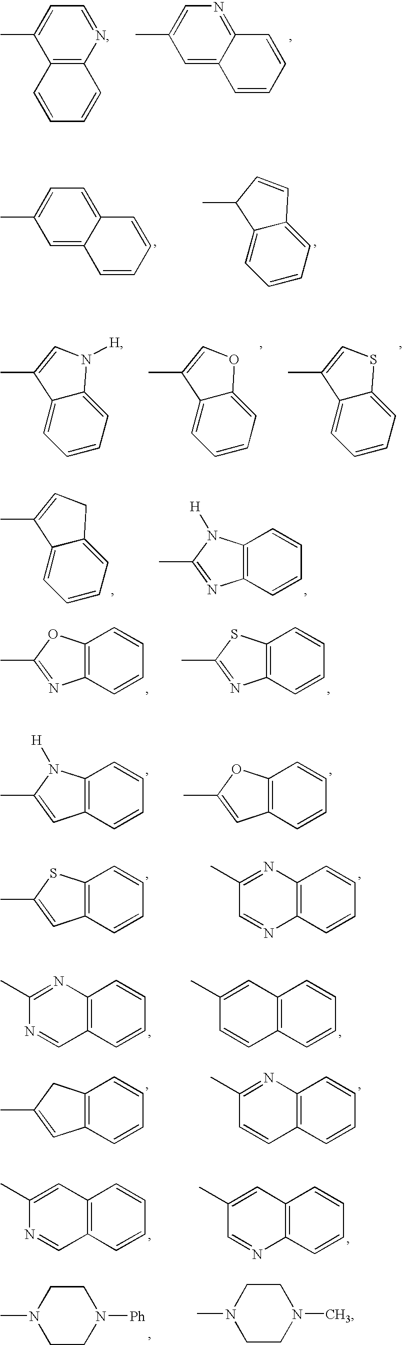

wherein the above-mentioned heteroaromatic and heterocyclic substituents can further bear one, two, three or more substituents selected from R⁵ R¹⁰;
R¹⁵, R¹⁷, R²⁰ represent independently of each other [symbol] ,

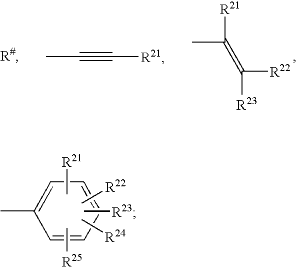

R⁵ R¹⁰, R¹² R²⁵ represent independently of each other R⁵⁰ R⁶⁹,

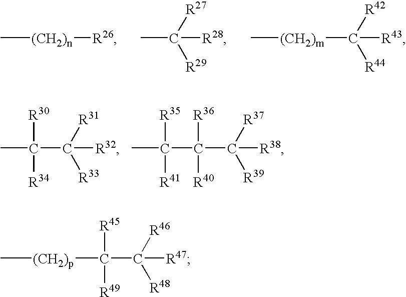

m, n, p are independently of each other integer from 0 10;
   and stereoisomeric forms, prodrugs, solvates, hydrates and/or pharmaceutically acceptable salts thereof,
   under the proviso that,
if R⁴ represents

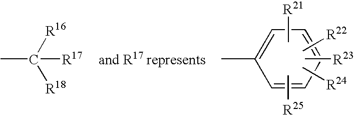

R¹⁶ is different from —H and —CH₃ or at least one of the substituents R²¹ R²⁵ is different from hydrogen.

For the sake of clarity,

R¹⁵, R¹⁷, R²⁰ represent independently of each other

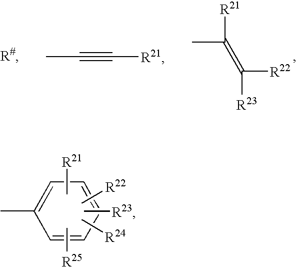

### R⁵⁰ R⁶⁹,

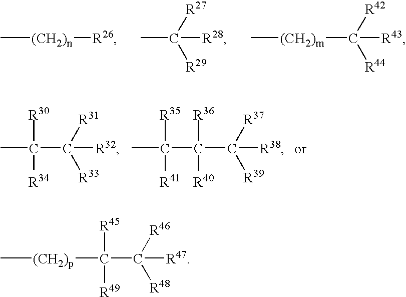

In particular embodiments, R¹ and/or R² are hydrogen.

In other embodiments, R³ is hydrogen.

The following subformula (II) (III) of formula (I) are especially preferred:

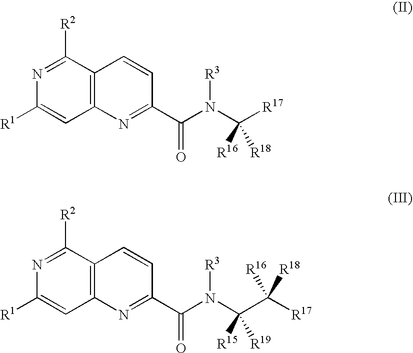

wherein
R¹ R [symbol] and R¹⁵ R¹⁹ have the meanings as defined above.

In particular embodiments, R¹⁷ in subformula (II) is thiophen-2-yl or thiophen-3-yl.

Further preferred are compounds according to subformula (II) wherein:

R¹, R², and R³ are hydrogen;
and one of R¹⁶ and R¹⁸ represents hydrogen and the other represents a substituent taken from the list of OH, OCH [symbol] , OC [symbol] H₅, OC [symbol] H₇, O cyclo-C₃H₅, OCH(CH [symbol] ), SH, SCH [symbol] , SC₂H₅, SC [symbol] H₇, S cyclo-C [symbol] H₅, SCH(CH₃)₂, SC(CH [symbol] )₃, NO [symbol] , F, Cl, Br, I, N [symbol] , CN, OCN, NCO, SCN, NCS, CHO, COCH [symbol] , COC₂H₅, COOH, COCN, COOCH [symbol] , COOC₂H₅, CONH [symbol] , CONHCH [symbol] , CONHC [symbol] H₅, CON(CH [symbol] )₂, NH [symbol] , NHCH₃, NHC₂H₅, NHC [symbol] H₇, NH cyclo-C [symbol] H₅, NHCH(CH₃)₂, SOCH₃, SOC₂H₅, SOC₈H₇, SO cyclo-C₃H₅, SOCH(CH [symbol] )₂, OCF₃, OC [symbol] F₅, —CH₂F —CHF₂, —CF₃, —CH₂Cl, —CHCl₂, —CCl₃, —CH₂Br —CHBr₂, —CBr₃, —CH₂—CH₂F —CH₂—CHF₂, —CH₂—CF₃, —CH₂—CH₂Cl, —CH₂—CHCl₂, —CH₂—CCl₃, —CH₂—CH₂Br —CH₂—CHBr₂, —CH₂—CBr₃, —CH₃, —C₂H₅, —C₃H₇, cyclo-C₈H₅, —CH(CH₃)₂, —C(CH₃)₃, —C₄H₉, —CH₂—CH(CH₃)₂, —CH(CH₃)—C₂H₅, —C(CH₃)₃, —CH═CH₂, —CH₂—CH═CH₂, —C(CH₃)═CH₂, —CH═CH—CH₃, C [symbol] H₄ CH—CH [symbol] , CH═C(CH₃)₂, —C≡CH, —C≡C—CH₃, and —CH₂≡C═CH;
and R¹⁷ has the meaning as defined above for compounds of formula (I).

In a certain embodiment wherein R¹⁷ is phenyl, R¹⁶ and R¹⁸ are not a combination of one hydrogen and one methyl substituent.

Further preferred are compounds according to subformula (II) wherein

R¹, R², and R³ are hydrogen;
and one of R¹⁶ and R¹⁸ represents hydrogen and the other represents a substituents taken from the list of CONH [symbol] , —CH₃, and —C₂H₅;
and R¹⁷ has the meaning as defined above for compounds of formula (I),

In a certain embodiment, wherein R¹⁷ is phenyl, R¹⁶ and R¹⁸ are not a combination of one hydrogen and one methyl substituent.

Preferably, R¹⁶ and R¹⁸ are a combination of one hydrogen and one ethyl substituent.

Further preferred are compounds according to subformula (III) wherein

R¹, R², and R³ are hydrogen;
and one of R¹⁵ and R¹⁹ represents hydrogen and the other represents a substituents taken from the list of OH, OCH [symbol] , OC [symbol] H₅, OC [symbol] H₇, O cyclo-C₃H₅, OCH(CH [symbol] ), SH, SCH [symbol] , SC₂H₅, SC [symbol] H₇, S cyclo-C [symbol] H₅, SCH(CH₃)₂, SC(CH [symbol] )₃, NO [symbol] , F, Cl, Br, I, N [symbol] , CN, OCN, NCO, SCN, NCS, CHO, COCH [symbol] , COC₂H₅, COOH, COCN, COOCH [symbol] , COOC₂H₅, CONH [symbol] , CONHCH [symbol] , CONHC [symbol] H₅, CON(CH [symbol] )₂, NH [symbol] , NHCH₃, NHC₂H₅, NHC [symbol] H₇, NH cyclo-C [symbol] H₅, NHCH(CH₃)₂, SOCH₃, SOC₂H₅, SOC₈H₇, SO cyclo-C₃H₅, SOCH(CH [symbol] )₂, OCF₃, OC [symbol] F₅, —CH₂F —CHF₂, —CF₃, —CH₂Cl, —CHCl₂, —CCl₃, —CH₂Br —CHBr₂, —CBr₃, —CH₂—CH₂F —CH₂—CHF₂, —CH₂—CF₃, —CH₂—CH₂Cl, —CH₂—CHCl₂, —CH₂—CCl₃, —CH₂—CH₂Br —CH₂—CHBr₂, —CH₂—CBr₃, —CH₃, —C₂H₅, —C₃H₇, cyclo-C₈H₅, —CH(CH₃)₂, —C(CH₃)₃, —C₄H₉, —CH₂—CH(CH₃)₂, —CH(CH₃)—C₂H₅, —C(CH₃)₃, —CH═CH₂, —CH₂—CH═CH₂, —C(CH₃)═CH₂, —CH═CH—CH₃, C [symbol] H₄ CH—CH [symbol] , CH═C(CH₃)₂, —C≡CH, —C≡C—CH₃, and —CH₂≡C═CH;
and R¹⁶, R¹⁷ and R¹⁸ have the meanings as defined above for compounds of formula (I).

Further preferred are compounds according to subformula (III) wherein

R¹, R², and R³ are hydrogen;
and one of R¹⁵ and R¹⁹ represents hydrogen and the other represents a substituents taken from the list of CONH₂, —CH₃, and —C₂H₅;
and R¹⁶, R¹⁷ and R¹⁸ have the meanings as defined above for compounds of formula (I).

Preferably, R¹⁶ and R¹⁸ are a combination of one hydrogen and one ethyl substituent.

In particular embodiments, the compounds according to subformulas (II) or (III) are present as racemic mixtures.

Especially preferred are all compounds in an enantiomeric or diastereomeric pure form, wherein the chiral center connected to the amid nitrogen has (R) or (S) configuration. Compounds of general formula (II) have one stereogenic center as far as the substituents do not contain further stereogenic centers. Enantiomers of said formula (II) are especially preferred, preferably having an ee larger than 90%, more preferably larger than 95% and especially preferred larger than 98%. Compounds of general formula (III) have two stereogenic centers as far as the substituents do not contain further stereogenic centers. Enantiomers and diastereomers of said formula (III) are especially preferred, preferably having an ee larger than 90%, a de larger than 90%, more preferably an ee larger than 95%, a de larger than 95%, and especially preferred an ee larger than 98% and a de larger than 98%, respectively.

Further preferred are compounds according to subformula (II) in an enantiomeric or diastereomeric pure form wherein:

R¹, R², R³ and R¹⁵ are hydrogen;
R¹⁶ represents OH, OCH [symbol] , OC [symbol] H₅, OC [symbol] H₇, O cyclo-C [symbol] H₅, OCH(CH [symbol] )₂, SH, SCH [symbol] , SC [symbol] H₅, SC [symbol] H₇, S cyclo-C₈H₅, SCH(CH₃)₂, SC(CH [symbol] )₃, NO [symbol] , F, Cl, Br, I, N [symbol] , CN, OCN, NCO, SCN, NCS, CHO, COCH₃, COC₂H₅, COOH, COCN, COOCH [symbol] , COOC [symbol] H₅, CONH [symbol] , CONHCH₃, CONHC [symbol] H₅, CON(CH [symbol] )₂, NH [symbol] , NHCH [symbol] , NHC [symbol] H₅, NHC₃H₇, NH cyclo-C [symbol] H₅, NHCH(CH [symbol] )₂, SOCH₃, SOC [symbol] H₅, SOC [symbol] H₇, SO cyclo-C [symbol] H₅, SOCH(CH [symbol] )₂, OCF [symbol] , OC [symbol] F₅, —CH₂F —CHF₂, —CF₃, —CH₂Cl, —CHCl₂, —CCl₃, —CH₂Br —CHBr₂, —CBr₃, —CH₂CH₂F —CH₂—CHF₂, —CH₂—CF₃, —CH₂—CH₂Cl, —CH—CHCl₂, —CH₂—CCl₃, —CH₂—CH₂Br —CH₂—CHBr₂, —CH₂—CBr₃, —CH₃, —C₂H₅, —C₃H₇, cyclo-C₃H₅, —CH(CH₃)₂, —C(CH₃)₃, —C₄H₉, —CH₂—CH(CH₃)₂, —CH(CH₃)—C₂H₅, —C(CH₃)₃, —CH═CH₂, —CH₂—CH═CH₂, —C(CH₃)═CH₂, —H═CH—CH₃, C [symbol] H₄ CH═CH [symbol] , CH═C(CH [symbol] )₂, —C≡CH, —C≡C—CH₃, —CH₂—C≡CH;
and R¹⁷ has the meaning as defined above for compounds of formula (I).

In a certain embodiment, wherein R¹⁶ is —CH₃, R¹⁷ is not phenyl.

Further preferred are compounds according to subformula (II) wherein

R¹, R², R³ and R¹⁸ are hydrogen;
R¹⁶ represents CONH [symbol] , —CH₃, or —C₂H₅;
and R¹⁷ has the meaning as defined above for compounds of formula (I),

In a certain embodiment, wherein R¹⁶ is —CH₃, R¹⁷ is not phenyl.

Preferably, R¹⁶ is —C₂H₅.

Further preferred are compounds according to subformula (III) wherein

R¹, R², R³ and R¹⁹ are hydrogen;
R¹⁵ represents OH, OCH [symbol] , OC [symbol] H₅, OC [symbol] H₇, O cyclo-C [symbol] H₅, OCH(CH [symbol] )₂, SH, SCH [symbol] , SC [symbol] H₅, SC [symbol] H₇, S cyclo-C₈H₅, SCH(CH₃)₂, SC(CH [symbol] )₃, NO [symbol] , F, Cl, Br, I, N [symbol] , CN, OCN, NCO, SCN, NCS, CHO, COCH₃, COC₂H₅, COOH, COCN, COOCH [symbol] , COOC [symbol] H₅, CONH [symbol] , CONHCH₃, CONHC [symbol] H₅, CON(CH [symbol] )₂, NH [symbol] , NHCH [symbol] , NHC [symbol] H₅, NHC₃H₇, NH cyclo-C [symbol] H₅, NHCH(CH [symbol] )₂, SOCH₃, SOC [symbol] H₅, SOC [symbol] H₇, SO cyclo-C [symbol] H₅, SOCH(CH [symbol] )₂, OCF [symbol] , OC [symbol] F₅, —CH₂F —CHF₂, —CF₃, —CH₂Cl, —CHCl₂, —CCl₃, —CH₂Br —CHBr₂, —CBr₃, —CH₂CH₂F —CH₂—CHF₂, —CH₂—CF₃, —CH₂—CH₂Cl, —CH—CHCl₂, —CH₂—CCl₃, —CH₂—CH₂Br —CH₂—CHBr₂, —CH₂—CBr₃, —CH₃, —C₂H₅, —C₃H₇, cyclo-C₃H₅, —CH(CH₃)₂, —C(CH₃)₃, —C₄H₉, —CH₂—CH(CH₃)₂, —CH(CH₃)—C₂H₅, —C(CH₃)₃, —CH═CH₂, —CH₂—CH═CH₂, —C(CH₃)═CH₂, —H═CH—CH₃, C [symbol] H₄ CH═CH [symbol] , CH═C(CH [symbol] )₂, —C≡CH, —C≡C—CH₃, —CH₂—C≡CH;
and R¹⁶, R¹⁷ and R¹⁸ have the meanings as defined above for compounds of formula (I).

Further preferred are compounds according to subformula (III) wherein

R¹, R², R³ and R¹⁹ are hydrogen;
R¹⁵ represents CONH [symbol] , —CH₃, or —C₂H₅;
and R¹⁶, R¹⁷ and R¹⁸ have the meanings as defined above for compounds of formula (I).

Preferably, R¹⁵ is —C₂H₅.

Furthermore, the following subformula (IIA) and (IIIA) of formula (I) are also especially preferred:

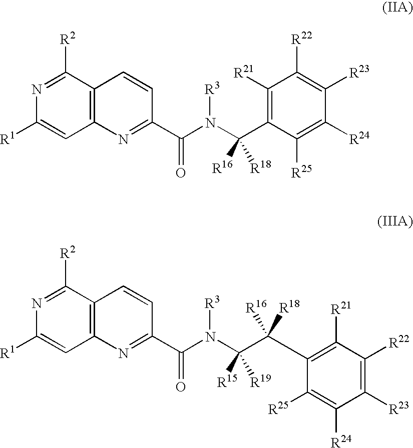

wherein
R¹ R³, R¹⁵, R¹⁶, R¹⁸, and R¹⁹ have the meanings as defined above for subformulas (II) and (III), and R²¹ R²⁵ have the meanings as defined above for formula (I).

In yet another preferred embodiment of the present invention the compound according to general formula (I) is selected from the group of compounds depicted in Table 1.

TABLE 1
Claimed compounds according to the present invention

| | | | LC ret. | MS | % inhi- |
|---|---|---|---|---|---|
| | | | time | [M + | bition |
| No. | MW | IUPAC name | [min] | H]+ | 10 μM |
| 1 | | [1,6]Naphthyridine-2- | | | 87 |
| | | carboxylic acid 3-bromo- | | | |
| | | benzylamide | | | |
| 2 | | [1,6]Naphthyridine-2- | | | |
| | | carboxylic acid 2-fluoro- | | | |
| | | benzylamide | | | |
| 3 | | [1,6]Naphthyridine-2- | | | |
| | | carboxylic acid 4-bromo- | | | |
| | | benzylamide | | | |
| 4 | | [1,6]Naphthyridine-2- | | | 81 |
| | | carboxylic acid (1-phenyl- | | | |
| | | ethyl)-amide | | | |
| 5 | | [1,6]Naphthyridine-2- | | | 62 |
| | | carboxylic acid (1-carb- | | | |
| | | amoyl-2-phenyl-ethyl)-amide | | | |
| 6 | | [1,6]Naphthyridine-2- | | | 65 |
| | | carboxylic acid [1- | | | |
| | | carbamoyl-2-(4-chloro- | | | |
| | | phenyl)-ethyl]-amide | | | |
| 7 | | [1,6]Naphthyridine-2- | | | 88 |
| | | carboxylic acid ((R)-1- | | | |
| | | phenyl-ethyl)-amide | | | |
| 8 | | [1,6]Naphthyridine-2- | | | 37 |
| | | carboxylic acid ((S)-1- | | | |
| | | phenyl-ethyl)-amide | | | |

Table 1 shows the HPLC-MS data for representative naphthyridine compounds and their inhibition rate (% inhibition of the protein kinase ROCK2) at a concentration of 10 μM.

In yet another embodiment of the present invention the compound according to formula (I) is [1,6]Naphthyridine-2-carboxylic acid thiophen-2-yl-methylamide.

In yet another embodiment of the present invention the compound according to formula (I) is selected from the list of compounds:
[1,6]Naphthyridine-2-carboxylic acid 1-phenyl-propylamide,
[1,6]Naphthyridine-2-carboxylic acid 1-(4-bromo-phenyl)-ethylamide,
[1,6]Naphthyridine-2-carboxylic acid 1-(4-chlorophenyl)-propylamide,
[1,6]Naphthyridine-2-carboxylic acid 1-(4-methylphenyl)-ethylamide,
[1,6]Naphthyridine-2-carboxylic acid 1-(4-chlorophenyl)-ethylamide,
[1,6]Naphthyridine-2-carboxylic acid 1-(4-methoxyphenyl)-ethylamide,
[1,6]Naphthyridine-2-carboxylic acid 1-(3-chlorophenyl)-propylamide,
[1,6]Naphthyridine-2-carboxylic acid 1-(4-fluorophenyl)-ethylamide,
[1,6]Naphthyridine-2-carboxylic acid 1-(4-methylphenyl)-propylamide,
[1,6]Naphthyridine-2-carboxylic acid 1-(thiophen-2-yl)-propylamide, and
[1,6]Naphthyridine-2-carboxylic acid 1-(thiophen-3-yl)-ethylamide,

In yet another embodiment of the present invention the compound according to formula (I) is selected from the list of compounds:
(R)-[1,6]Naphthyridine-2-carboxylic acid (1-phenyl-ethyl)-amide,
(S)-[1,6]Naphthyridine-2-carboxylic acid (1-carbamoyl-2-phenyl-ethyl)-amide,
(S)-[1,6]Naphthyridine-2-carboxylic acid [1-carbamoyl-2-(4-hydroxy-phenyl)-ethyl]-amide,
(R)-[1,6]Naphthyridine-2-carboxylic acid (1-phenyl-ethyl)-amide,
(R)-[1,6]Naphthyridine-2-carboxylic acid 1-(naphth-2-yl)-ethylamide,
(R)-[1,6]Naphthyridine-2-carboxylic acid 1-(4-methylphenyl)-ethylamide,
(R)-[1,6]Naphthyridine-2-carboxylic acid 1-phenyl-propylamide, and
(R)-[1,6]Naphthyridine-2-carboxylic acid 1-(4-chloro-phenyl)-ethylamide.

The present invention also comprises pharmaceutically acceptable salts of the compounds according to the general formula (I), all stereoisomeric forms of the compounds according to the general formula (I) as well as solvates, especially hydrates or prodrugs thereof. A prodrug is commonly described as an inactive or protected derivative of an active ingredient or a drug, which is converted to the active ingredient or drug in the body.

The compounds of the present invention are basic and may form salts with organic or inorganic acids. Examples of suitable acids for such acid addition salt formation are hydrochloric acid, hydrobromic acid, sulfuric acid, phosphoric acid, acetic acid, citric acid, oxalic acid, malonic acid, salicylic acid, p-aminosalicylic acid, malic acid, fumaric acid, succinic acid, ascorbic acid, maleic acid, sulfonic acid, phosphonic acid, perchloric acid, nitric acid, formic acid, propionic acid, gluconic acid, lactic add, tartaric acid, hydroxymaleic acid, pyruvic acid, phenylacetic acid, benzoic acid, p-aminobenzoic acid, p-hydroxybenzoic acid, methanesulfonic acid, ethanesulfonic acid, nitrous acid, hydroxyethanesulfonic acid, ethylenesulfonic acid, p-toluenesulfonic acid, naphthylsulfonic acid, sulfanilic acid, camphorsulfonic acid, china acid, mandelic acid, o-methylmandelic acid, hydrogen-benzenesulfonic acid, picric acid, adipic acid, d-o-tolyltartaric acid, tartronic acid, (o, m, p)-toluic acid, naphthylamine sulfonic acid, and other mineral or carboxylic acids well known to those skilled in the art. The salts are prepared by contacting the free base form with a sufficient amount of the desired acid to produce a salt in the conventional manner. In case, the compounds bear acidic substituents, the formation of salts with inorganic or organic bases may be possible. Examples for such bases are NaOH, KOH, NH₄OH, tetraalkylammonium hydroxide, lysine or arginine and the like. Salts may be prepared in a conventional manner using methods well known in the art, for example by treatment of a solution of the compound of the general formula (I) with a solution of an acid, selected out of the group mentioned above.

Some of the compounds of the present invention may be crystallised or recrystallised from solvents such as aqueous and organic solvents. In such cases solvates may be formed. This invention includes within its scope stoichiometric solvates including hydrates as well as compounds containing variable amounts of water that may be produced by processes such as lyophilisation.

Certain compounds of the general formula (I) may exist in the form of optical isomers, e.g. enantiomers, diastereoisomers and mixtures of isomers in all ratios, e.g. racemic mixtures. The invention includes all such forms, in particular the pure isomeric forms. The different isomeric forms may be separated or resolved one from the other by conventional methods, or any given isomer may be obtained by conventional synthetic methods or by stereospecific or asymmetric syntheses. Where a compound according to the general formula (I) contains an alkene moiety, the alkene can be presented as a cis or trans isomer or a mixture thereof. When an isomeric form of a compound of the invention is provided substantially free of other isomers, it will preferably contain less than 5% w/w, more preferably less than 2% w/w and especially less than 1% w/w of the other isomer(s). The afore-mentioned compounds are useful as pharmaceutically active agents, i.d. as drugs or medicine.

Still another aspect of the present invention deals with pharmaceutical compositions comprising at least one compound according to general formula (I) as an active ingredient, together with at least one pharmaceutically acceptable carrier, excipient and/or diluents. The pharmaceutical compositions of the present invention can be prepared in a conventional solid or liquid carrier or diluents and a conventional pharmaceutically-made adjuvant at suitable dosage level in a known way. The preferred preparations are adapted for oral application. These administration forms include, for example, pills, tablets, film tablets, coated tablets, capsules, liposomal formulations, micro- and nano-formulations, powders and deposits.

Furthermore, the present invention also includes pharmaceutical preparations for parenteral application, including dermal, intradermal, intragastral, intracutan, intravasal, intravenous, intramuscular, intraperitoneal, intranasal, intravaginal, intrabuccal, percutan, rectal, subcutaneous, sublingual, topical, or transdermal application, which preparations in addition to typical vehicles and/or diluents contain at least one compound according to the present invention and/or a pharmaceutical acceptable salt thereof as active ingredient.

The pharmaceutical compositions according to the present invention containing at least one compound according to the present invention, especially one pure optical isomer, and/or a pharmaceutically acceptable salt thereof as active ingredient will typically be administered together with suitable carrier materials selected with respect to the intended form of administration, i.e. for oral administration in the form of tablets, capsules (either solid filled, semi-solid filled or liquid filled), powders for constitution, aerosol preparations consistent with conventional pharmaceutical practices. Other suitable formulations are gels, elixirs, dispersable granules, syrups, suspensions, creams, lotions, solutions, emulsions, suspensions, dispersions, and the like. Suitable dosage forms for sustained release include tablets having layers of varying disintegration rates or controlled release polymeric matrices impregnated with the active components and shaped in tablet form or capsules containing such impregnated or encapsulated porous polymeric matrices.

As pharmaceutically acceptable carrier, excipient and/or diluents can be used carriers such as preferably with an inert carrier like lactose, starch, sucrose, cellulose, magnesium stearate, dicalcium phosphate, calcium sulfate, talc, mannitol, ethyl alcohol (liquid filled capsules);

suitable binders include starch, gelatin, natural sugars, corn sweeteners, natural and synthetic gums such as acacia, sodium alginate, carboxymethylcellulose, polyethylene glycol and waxes, sugars such as sucrose, starches derived from wheat corn rice and potato, natural gums such as acacia, gelatin and tragacanth, derivatives of seaweed such as alginic acid, sodium alginate and ammonium calcium alginate, cellulose materials such as methylcellulose, sodium carboxymethylcellulose and hydroxypropylmethylcellulose, polyvinylpyrrolidone, and inorganic compounds such as magnesium aluminum silicate;
lubricants such as boric acid, sodium benzoate, sodium acetate, sodium chloride, magnesium stearate, calcium stearate, or potassium stearate, stearic acid, high melting point waxes, and other water soluble lubricants such as sodium chloride, sodium benzoate, sodium acetate, sodium oleate, polyethylene glycols and D,L-leucine;
disintegrating agents (disintegrates) such as starch, methylcellulose, guar gum, modified starches such as sodium carboxymethyl starch, natural and synthetic gums such as locust bean, karaya, guar, tragacanth and agar, cellulose derivatives such as methylcellulose and sodium carboxymethylcellulose, microcrystalline celluloses, and cross-linked microcrystalline celluloses such as sodium croscarmellose, alginates such as alginic acid and sodium alginate, clays such as bentonites, and effervescent mixtures;
coloring agents, sweetening agents, flavoring agents, preservatives; glidents are for example silicon dioxide and talc; suitable adsorbent are clay, aluminum oxide,
suitable diluents are water or water/propylene glycol solutions for parenteral injections, juice, sugars such as lactose, sucrose, mannitol, and sorbitol, starches derived from wheat, corn rice, and potato, and celluloses such as microcrystalline cellulose.

Another aspect of the present invention relates to the use of the compounds according to general formula (I)

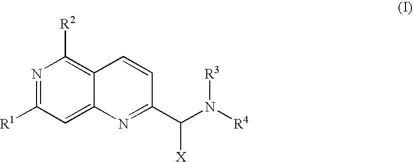

wherein
R¹, R², R²⁶ R⁶⁹ represent independently of each other H, OH, OCH [symbol] , OC [symbol] H₅, OC [symbol] H₇, O cyclo-C [symbol] H₅, OCH(CH [symbol] )₂, OC(CH [symbol] )₃, OC [symbol] H₉, —OPh, —OCH₂-Ph, —OCPh₃, SH, SCH [symbol] , SC₂H₅, SC [symbol] H₇, S cyclo-C [symbol] H₅, SCH(CH [symbol] )₂, SC(CH [symbol] )₃, NO [symbol] , F, Cl, Br, I, N [symbol] , CN, OCN, NCO, SCN, NCS, CHO, COCH [symbol] , COC [symbol] H₅, COC [symbol] H₇, CO cyclo-C [symbol] H₅, COCH(CH [symbol] )₂, COC(CH [symbol] )₃, COOH, COCN, COOCH [symbol] , COOC [symbol] H₅, COOC [symbol] H₇, COO cyclo-C [symbol] H₅, COOCH(CH [symbol] )₂, COOC(CH [symbol] )₃, OOC CH(CH [symbol] )₂, OOC C [symbol] H₅, OOC C [symbol] H₇, OOC cyclo-C [symbol] H₅, OOC CH(CH [symbol] )₂, OOC C(CH [symbol] )₃, CONH [symbol] , CONHCH [symbol] , CONHC [symbol] H₅, CONHC [symbol] H₇, CONH cyclo-C [symbol] H₅, CONH[CH(CH [symbol] )₂], CONH[C(CH [symbol] )₃], CON(CH [symbol] )₂, CON(C [symbol] H₅)₂, CON(C [symbol] H₇)₂, CON(cyclo-C [symbol] H₅)₂, CON[CH(CH [symbol] )₂]₂, CON[C(CH [symbol] )₃]₂, NH₂, NHCH [symbol] , NHC [symbol] H₅, NHC [symbol] H₇, NH cyclo-C [symbol] H₅, NHCH(CH [symbol] )₂, NHC(CH [symbol] )₃, N(CH₃)₂, N(C [symbol] H₅)₂, N(C₆H₇)₂, N(cyclo-C [symbol] H₅)₂, N[CH(CH₃)₂]₂, N[C(CH [symbol] )₃]₂, SOCH [symbol] , SOC [symbol] H₅, SOC [symbol] H₇, SO cyclo-C [symbol] H₅, SOCH(CH [symbol] )₂, SOC(CH [symbol] )₃, SO [symbol] CH₃, SO [symbol] C₂H₅, SO₂C₃H₇, SO [symbol] cyclo-C [symbol] H₅, SO [symbol] CH(CH₃)₂, SO [symbol] C(CH₃)₃, SO [symbol] H, SO [symbol] CH₃, SO₃C₂H₅, SO [symbol] C₃H₇, SO [symbol] cyclo-C [symbol] H₅, SO [symbol] CH(CH₃)₂, SO [symbol] C(CH₃)₃, OCF [symbol] , OC₂F₅, O COOCH [symbol] , O COOC [symbol] H₅, O COOC [symbol] H₇, O COO cyclo-C [symbol] H₅, O COOCH(CH [symbol] )₂, O COOC(CH [symbol] )₃, NH CO NH [symbol] NH CO NHCH [symbol] , NH CO NHC [symbol] H₅, NH CO NHC [symbol] H₇, NH CO NH cyclo-C [symbol] H₅, NH CO NH[CH(CH [symbol] ) [symbol] ], NH CO NH[C(CH [symbol] )₃], NH CO N(CH [symbol] )₂, NH CO N(C [symbol] H₅)₂, NH CO N(C [symbol] H₇)₂, NH CO N(cyclo-C [symbol] H₅)₂, NH CO N[CH(CH [symbol] )₂]₂, NH CO N[C(CH [symbol] )₃]₂, NH CS NH [symbol] , NH CS NH cyclo-C [symbol] H [symbol] , NH CS NHC [symbol] H₇, NH CS NH[CH(Ch [symbol] )₂], NH CS NH[C(CH [symbol] )₃], NH CS N(CH [symbol] )₂, NH CS N(CH [symbol] )₂, NH CS N(C [symbol] H₇)₂, NH CS N(cyclo-C [symbol] H₅)₂, NH C(═NH) NH [symbol] , NH CS NHC [symbol] H₅, NH CS NHCH [symbol] , O CO NH [symbol] [symbol] . O CO NHCH [symbol] , O CO NH [symbol] H₅, O CO NHC [symbol] H₇, O CO NH cyclo-C [symbol] H₅, O CO NH[CH(CH [symbol] )₂], O CO NH[C(CH [symbol] )₃], O CO N(CH [symbol] )₂, O CO N(C [symbol] H₅)₂, O CO N(C [symbol] H₇)₂, O CO N(cyclo-C [symbol] H₅)₂, O CO N[CH(CH(CH [symbol] )₂]₂, O CO N[C(CH [symbol] )₃]₂, O CO OCH [symbol] , O CO OC [symbol] H₅, O CO OC [symbol] H₇, O CO O cyclo-C [symbol] H₅, O CO OCH(CH [symbol] )₂, O CO OC(CH [symbol] )₃, —CH₂F —CHF₂, —CF₃, —CH₂Cl, —CHCl₂, —CCl₃, —CH₂Br—CHBr₂, —CBr₃, —CPh₃, —CH₂—CH₂F—CH₂—CHF₂, —CH₂—CF₃, —CH₂—CH₂Cl, —CH₂—CHCl₂, —CH₂—CCl₃, —CH₂—CH₂Br—CH₂—CHBr₂, —CH₂—CBr₃, —CH₃, —C₂H₅, —C₃H₇, cyclo-C [symbol] H₅, —CH(CH₃)₂, —C(CH₃)₃, —C₄H₉, —CH₂—CH(CH₃)₂, —CH(CH₃)—C₂H₅, —C(CH₃)₃, —C₅H₁₁, —C₆H₁₃, —C₇H₁₅, —C₈H₁₇, —C₉H₁₉, —C₁₀H₂₁, -Ph, —CH₂-Ph, —CH═CH₂, —CH₂—CH═CH₂, —C(CH₃)═CH₂, —CH═CH—CH₃, C [symbol] H₄ CH═CH [symbol] , CH═C(CH [symbol] )₂, —C≡CH, —C≡C—CH₃, —CH₂—C≡CH;
R³, R¹¹ represent independently of each other —H, —CH₃, —C₂H₅, —C₃H₇, cyclo-C [symbol] H₅, —CH(CH₃)₂, —C(CH₃)₃, —C₄H₉, —CH₂—CH(CH₃)₂, —CH(CH₃)—C₂H₅, —C(CH₃)₃, —C₅H₁₁, —C₆H₁₃, —C₇H₁₅, —C₈H₁₇, —C₉H₁₉, —C₁₀H₂₁, -Ph, —CH₂-Ph, —CH═CH₂, —CH₂—CH═CH₂, —C(CH₃)═CH₂, —CH═CH—CH₃, C [symbol] H₄ CH═CH [symbol] , CH═C(CH [symbol] )₂, —C≡CH, —C≡C—CH₃, —CH₂C≡CH;
the moiety

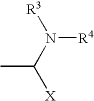

represents

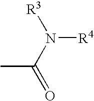

### or R*;

R⁴ represents R** or

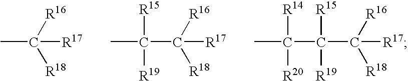

R* and R** represent independently of each other

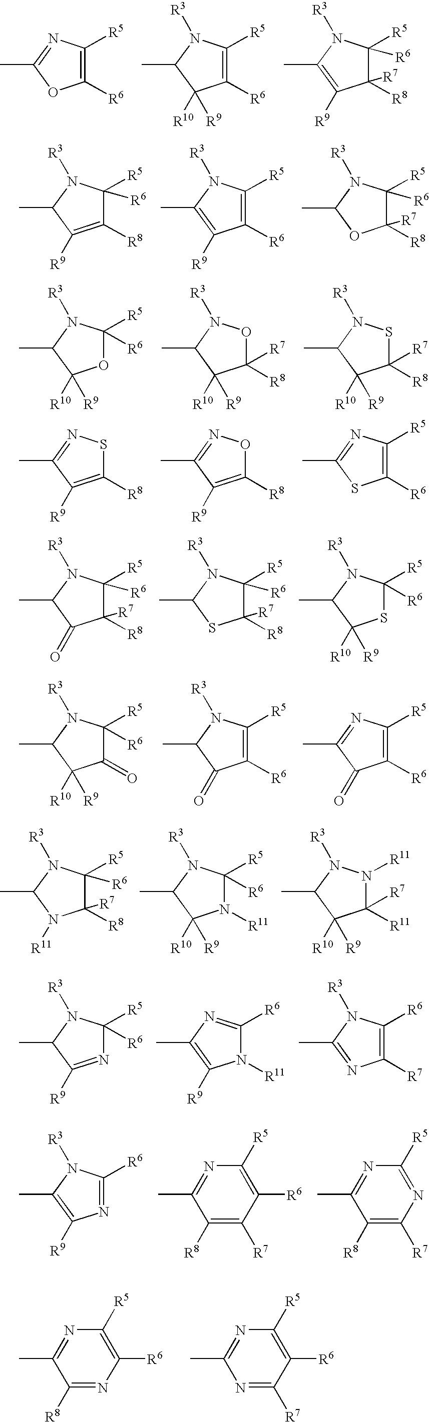

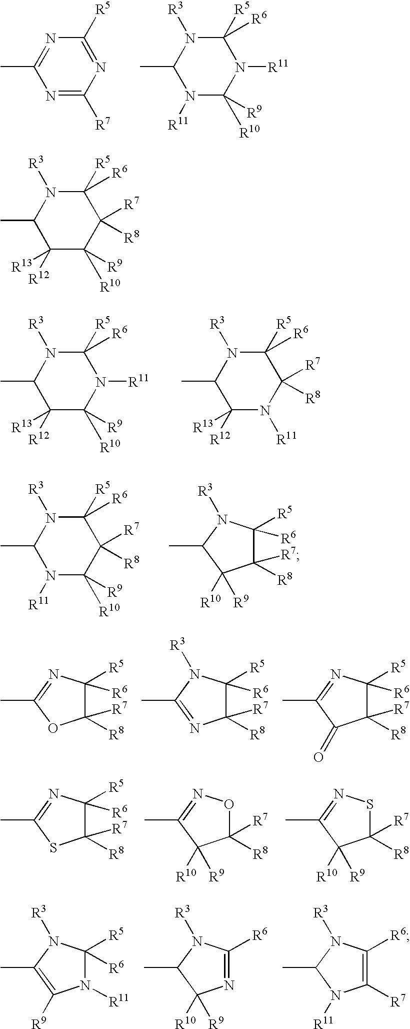

R# represents R** or the above-mentioned heteroaromatic or heterocyclic residues, wherein the above-mentioned heteroaromatic and heterocyclic substituents can further bear one, two, three or more substituents selected from R⁵ R¹⁰;
R¹⁵, R¹⁷, R²⁰ represent independently of each other

[symbol]

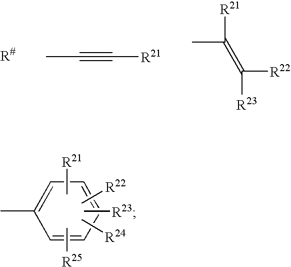

R⁵ R¹⁰, R¹² R²⁵ represent independently of each other R⁵⁰ R⁶⁹,

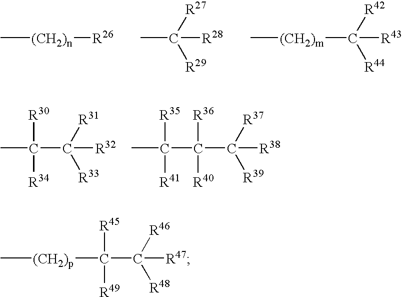

m, n, p are independently of each other integer from 0 10;
and stereoisomeric forms, prodrugs, solvates, hydrates and/or pharmaceutically acceptable salts thereof as inhibitor of the protein kinases ROCK1 and/or ROCK2.

Many aspects of cellular behaviour, like motility, morphology, cell-cycle progression or cell differentiation, involve the actin cytoskeleton, which consists of a meshwork of actin filaments and actin-binding proteins. It is controlled by the Rho family of small monomeric GTPases. The most intensively studied members Cdc42, Rac, and Rho govern the dynamics of the actin cytoskeleton to drive, for example, the movement of a cell in an organized and directed way. Various agonists (e.g. thrombin, lysophosphatidic acid, sphingosine-1-phosphate, or thromboxane A2), which signal through G protein-coupled receptors can lead to the activation of these small GTPases, which then transduce the signal to downstream effectors. GTPases are molecular switches that cycle between an inactive (GDP-bound) and an active (GTP-bound) form.

RhoA was found to bind and activate a variety of effectors: e.g. ROCK (Rho-associated coiled-coil containing kinase), protein kinase N, rhophilin, rhotekin, citron kinase, PIP5 kinase, or phospholipase D. Among those effectors Rho-kinases play a major role in the arrangement of the actin cytoskeleton. Two isoforms of ROCK have been identified: ROCK1 (ROCK I, ROKβ, p160ROCK) and ROCK2 (ROCK II, ROKα). They are serine/threonine kinases of ˜160 kDa with an amino-terminal kinase domain, followed by a coiled-coil region with the Rho-binding domain and a carboxy-terminal pleckstrin homology domain that acts as an autoregulatory domain. Both isoforms are ubiquitously expressed and share ˜92% identity in their kinase domains. So far, different activities between the two isoforms have not been identified. ROCKs belong to the AGC class of serine/threonine kinases and are homologous to DMPK (myotonic dystrophy kinase), MRCK (Cdc42-binding kinase) and citron kinase.

Upon binding of RhoA the carboxy-terminus of ROCK opens up from the kinase domain and the inhibitory effect on the kinase activity is released. Besides RhoA, arachidonic acid can activate ROCKs in a Rho-independent way. Constructs of ROCKs, which lack the carboxy-terminal inhibitory domain result in constitutively active kinases. In cells, caspase-3 cleaves ROCK I during apoptosis and turns ROCK I into an active kinase.

ROCKs phosphorylate a variety of substrates: myosin binding subunit of the myosin light chain phosphatase, myosin light chain kinase, myosin light chain, LIM kinase 1 and 2, ERM proteins (ezrin, radixin, moesin), adducin, the sodium-hydrogen exchanger NHE1, vimentin, GFAP (glial fibrillary acidic protein, NF-L (neurofilament L protein), CRMP1 and 2 (collapsing response mediator protein 2), eukaryotic elongation factor 1α, calponin, CD44, and IP3 receptors. Most of these targets are involved in regulating the actin-filament assembly.

For cellular contractility the major determinant is the phosphorylation state of myosin II, which depends on the counteracting activities of MLCK (myosin light chain kinase) and MLC phosphatase (MLCP). Whereas MLCK-mediated phosphorylation of myosin II occurs under high Ca²⁺ concentrations, a so-called “Ca²⁺-independent pathway exists, which is due to an inactivation of MLC phosphatase. This inactivation process relies on the phosphorylation of MBS (myosin binding subunit) of MLC phosphatase by ROCK. Thereby the amount of phosphorylated myosin II and subsequently actomyosin crossbridging is increased leading to a contraction of the actin cytoskeleton. As a result in smooth muscle cells ROCK induces contraction, in non-muscle cells stress fibre formation and focal adhesions, and in neuronal cells neurite retraction.

Moreover, another pathway involving ROCK has a major stabilizing impact on the actin cytoskeleton. ROCKs phosphorylate LIM kinases 1 and 2, which in turn phosphorylate cofilin. Cofilin is an actin-binding protein, which causes in its un-phosphorylated state de-polymerization of actin filaments. Phosphorylation of cofilin by LIM kinases, however, increases and stabilizes the number of actin filaments (Van Aelst, L & D'Souza-Schorey, C. (1997). Genes Dev. 11: 2295-2322).

Due to their pivotal role in the rearrangement of the actin cytoskeleton ROCKs have been shown to mediate cellular contraction, cell adhesion, cell migration, invasion, cell shape, cell size, cell differentiation, phagocytosis, apoptosis, neurite rectraction, and cytokinesis.

Experiments with inhibitors against ROCK proof the involvement of ROCK in these processes. The most evaluated chemical inhibitors against ROCK are the pyridine derivatives Y-27632 and Y-30141, as well as the isoquinolinesulfonamides HA-1077, H9, H89. However, none of these inhibitors displays a complete selectivity against ROCK. These compounds also inhibit the kinase activities of PKA, PKC, PKN, and citron kinase. Y-27632 was used to demonstrate the role of ROCK in various processes in vivo: tumour cell growth and metastasis, erectile dysfunction, cerebral vasospasm, coronary vasospasm, atherosclerosis, hypertension, bronchial asthma, myocardial hypertrophy, neuronal degeneration and spinal cord injuries, preterm labor, platelet aggregation, leukocyte aggregation, intraocular pressure, and bone resorption. Fasudil was approved for the treatment of cerebral vasospasm and ischemia following subarachnoid hemorrhage in humans (Narumiya, S, Ishizaki, T, and Uehata, M (2000). Methods in Enzymol. 325: 273-284).

Thus, the compounds mentioned herein are useful for prophylaxis and/or treatment of diseases associated with the enzymes ROCK1 and/or ROCK2 or curable by inhibition of the enzymes ROCK1 and/or ROCK2. Preferred are the pure optical isomers, e.g. enantiomers and diastereomers as mentioned above.

Especially, the compounds mentioned herein can be used for prophylaxis and/or treatment of cancers, inflammation, infectious diseases, HIV, erectile dysfunction, cardiovascular diseases and disorders, hypertension, angina pectoris, cerebral ischaemia, cerebral vasospasm, myocardial ischaemia, coronary vasospasm, heart failure, myocardial hypertrophy, atherosclerosis, restenosis, spinal cord injuries, neuronal degeneration, thrombotic disorders, asthma, glaucoma, and osteoporosis.

Accordingly, another aspect of the invention relates to the use of the compounds mentioned herein for the preparation of a medicament for the treatment of a disease selected from the list of cancers, inflammation, infectious diseases, HIV, erectile dysfunction, cardiovascular diseases and disorders, hypertension, angina pectoris, cerebral ischaemia, cerebral vasospasm, myocardial ischaemia, coronary vasospasm, heart failure, myocardial hypertrophy, atherosclerosis, restenosis, spinal cord injuries, neuronal degeneration, thrombotic disorders, asthma, glaucoma, and osteoporosis

In a particular embodiment, the use of the compounds mentioned herein is for the preparation of a medicament for the treatment of a disease that is not taken from the list of HIV, HBV, HCV, HSV-1, HSV-2, Parainfluenza, Influenza A, Influenza B, Adenovirus, RV and RVS. In yet another particular embodiment, the use of the compounds mentioned herein is for the preparation of a medicament for the treatment of a disease that is not taken from the list of infectious diseases and HIV.

Various cancer types and tumors can be treated by the compounds according to general formula (I) such as adenocarcinoma, choroidal melanoma, acute leukemia, acoustic neurinoma, ampullary carcinoma, anal carcinoma, astrocytoma, basal cell carcinoma, pancreatic cancer, desmoid tumor, bladder cancer, bronchial carcinoma, breast cancer, Burkitt's lymphoma, corpus cancer, CUP-syndrome (carcinoma of unknown primary), colorectal cancer, small intestine cancer, small intestinal tumors, ovarian cancer, endometrial carcinoma, ependymoma, epithelial cancer types, Ewing's tumors, gastrointestinal tumors, gastric cancer, gallbladder cancer, gall bladder carcinomas, uterine cancer, cervical cancer, cervix, glioblastomas, gynecologic tumors, ear, nose and throat tumors, hematologic neoplasias, hairy cell leukemia, urethral cancer, skin cancer, skin testis cancer, brain tumors (gliomas), brain metastases, testicle cancer, hypophysis tumor, carcinoids, Kaposi's sarcoma, laryngeal cancer, germ cell tumor, bone cancer, colorectal carcinoma, head and neck tumors (tumors of the ear, nose and throat area), colon carcinoma, craniopharyngiomas, oral cancer (cancer in the mouth area and on lips), cancer of the central nervous system, liver cancer, liver metastases, leukemia, eyelid tumor, lung cancer, lymph node cancer (Hodgkin's/Non-Hodgkin's), lymphomas, stomach cancer, malignant melanoma, malignant neoplasia, malignant tumors gastrointestinal tract, breast carcinoma, rectal cancer, medulloblastomas, melanoma, meningiomas, Hodgkin's disease, mycosis fungoides, nasal cancer, neurinoma, neuroblastoma, kidney cancer, renal cell carcinomas, non-Hodgkin's lymphomas, oligodendroglioma, esophageal carcinoma, osteolytic carcinomas and osteoplastic carcinomas, osteosarcomas, ovarial carcinoma, pancreatic carcinoma, penile cancer, plasmocytoma, prostate cancer, pharyngeal cancer, rectal carcinoma, retinoblastoma, vaginal cancer, thyroid carcinoma, Schneeberger disease, esophageal cancer, spinalioms, T-cell lymphoma (mycosis fungoides), thymoma, tube carcinoma, eye tumors, urethral cancer, urologic tumors, urothelial carcinoma, vulva cancer, wart appearance, soft tissue tumors, soft tissue sarcoma, Wilm's tumor, cervical carcinoma and tongue cancer.

Also several infectious diseases and opportunistic infections can be treated by the compounds mentioned herein such as AIDS, Alveolar Hydatid Disease (AHD, Echinococcosis), Amebiasis (Entamoeba histolytica Infection), Angiostrongylus Infection, Anisakiasis, Anthrax, Babesiosis (Babesia Infection), Balantidium Infection (Balantidiasis), Baylisascaris Infection (Raccoon Roundworm), Bilharzia (Schistosomiasis), Blastocystis hominis Infection (Blastomycosis), Boreliosis, Botulism, Brainerd Diarrhea, Brucellosis, BSE (Bovine Spongiform Encephalopathy), Candidiasis, Capillariasis (Capillaria Infection), CFS (Chronic Fatigue Syndrome), Chagas Disease (American Trypanosomiasis), Chickenpox (Varicella-Zoster virus), Chiamydia pneumoniae Infection, Cholera, Chronic Fatigue Syndrome, CJD (Creutzfeldt-Jakob Disease), Clonorchiasis (Clonorchis Infection), CLM (Cutaneous Larva Migrans, Hookworm Infection), Coccidioidomycosis, Conjunctivitis, Coxsackievirus A16 (Hand, Foot and Mouth Disease), Cryptococcosis, Cryptosporidium Infection (Cryptosporidiosis), Culex mosquito (Vector of West Nile Virus), Cutaneous Larva Migrans (CLM), Cyclosporiasis (Cyclospora Infection), Cysticercosis (Neurocysticercosis), Dengue/Dengue Fever, Ebola Virus Hemorrhagic Fever, Echinococcosis (Alveolar Hydatid Disease), Encephalitis, Entomoeba coli Infection, Entomoeba dispar Infection, Entomoeba hartmanni Infection, Entomoeba histolytica Infection (Amebiasis), Entomoeba polecki Infection, Enterobiasis (Pinworm Infection), Enterovirus Infection (Non-Polio), Epstein-Barr Virus Infection, Escherichia coli Infection, Foodborne Infection, Foot and mouth Disease, Fungal Dermatitis, Gastroenteritis, Group A streptococcal Disease, Group B streptococcal Disease, Hansen's Disease (Leprosy), Hantavirus Pulmonary Syndrome, Helicobacter pylori Infection, Hematologic Disease, Hendra Virus Infection, Hepatitis (HCV, HBV), HIV Infection, Human Ehrlichiosis, Human Parainfluenza Virus Infection, Influenza, Isosporiasis (Isospora Infection), Lassa Fever, Leishmaniasis, Kala-azar (Kala-azar, Leishmania Infection), Leprosy, Lice (Body lice, Head lice, Pubic lice), Lyme Disease, Malaria, Marburg Hemorrhagic Fever, Measles, Meningitis, Mosquito-borne Diseases, Mycobacterium avium Complex (MAC) Infection, Naegleria Infection, Nosocomial Infections, Nonpathogenic Intestinal Amebae Infection, Onchocerciasis (River Blindness), Opisthorciasis (Opisthorcis Infection), Parvovirus Infection, Plague, PCP (Pneumocystis carinii Pneumonia), Polio, Q Fever, Rabies, Respiratory Syncytial Virus (RSV) Infection, Rheumatic Fever, Rift Valley Fever, River Blindness (Onchocerciasis), Rotavirus Infection, Roundworms Infection, Salmonellosis, Salmonella Enteritidis, Scabies, Shigellosis, Shingles, Sleeping Sickness, Smallpox, Streptococcal Infection, Tapeworm Infection (Taenia Infection), Tetanus, Toxic Shock Syndrome, Tuberculosis, Ulcers (Peptic Ulcer Disease), Valley Fever, Vibrio parahaemolyticus Infection, Vibrio vulnificus Infection, Viral Hemorrhagic Fever, Warts, Waterborne infectious Diseases, West Nile Virus Infection (West Nile Encephalitis), Whooping Cough, Yellow Fever, tuberculosis, leprosy, mycobacteria-induced meningitis, wherein Cytomegalovirus Infection is excluded.

Another aspect of the present invention is directed to the use of at least one compound of any one of the general formulas (I), (II), (IIA), (III) or (IIIA), optical isomers and/or pharmaceutically acceptable salts thereof for prophylaxis and/or treatment of cardiovascular diseases and cardiovascular disorders such as adult congenital heart disease, aneurysm, stable angina, unstable angina, angina pectoris, angioneurotic edema, aortic valve stenosis, aortic aneurysm, arrhythmia, arrhythmogenic right ventricular dysplasia, arteriosclerosis, arteriovenous malformations, atrial fibrillation, Behcet syndrome, bradycardia, cardiac tamponade, cardiomegaly, congestive cardiomyopathy, hypertrophic cardiomyopathy, restrictive cardiomyopathy, cardiovascular disease prevention, carotid stenosis, cerebral hemorrhage, Churg-Strauss syndrome, diabetes, Ebstein's Anomaly, Eisenmenger complex, cholesterol embolism, bacterial endocarditis, fibromuscular dysplasia, congenital heart defects, heart diseases, congestive heart failure, heart valve diseases, heart attack, epidural hematoma, hematoma, subdural, Hippel-Lindau disease, hyperemia, hypertension, pulmonary hypertension, hypertrophic growth, left ventricular hypertrophy, right ventricular hypertrophy, hypoplastic left heart syndrome, hypotension, intermittent claudication, ischemic heart disease, Klippel-Trenaunay-Weber syndrome, lateral medullary syndrome, long QT syndrome mitral valve prolapse, moyamoya disease, mucocutaneous lymph node syndrome, myocardial infarction, myocardial ischemia, myocarditis, pericarditis, peripheral vascular diseases, phlebitis, polyarteritis nodosa, pulmonary atresia, Raynaud disease, restenosis, Sneddon syndrome, stenosis, superior vena cava syndrome, syndrome X, tachycardia, Takayasu's arteritis, hereditary hemorrhagic telangiectasia, telangiectasis, temporal arteritis, tetralogy of fallot, thromboanguitis obliterans, thrombosis, thromboembolism, tricuspid atresia, varicose veins, vascular diseases, vasculitis, vasospasm, ventricular fibrillation, Williams syndrome, peripheral vascular disease, varicose veins and leg ulcers, deep vein thrombosis, Wolff-Parkinson-White syndrome.

Another important aspect of the present invention deals with the use of the decaline-derived compounds in combination with common drugs such as anti-HIV drugs, antiproliferative drug, cytotoxic or cytostatic drug, ganciclovir, foscamet, cidofovir, valganciclovir, fomivirsen, penciclovir or valaciclovir. In some cases, the inventive compounds are able to increase the activity of the common drugs and/or reduce their undesired side effects.

### DESCRIPTION OF FIGURES

FIG. 1 shows the general subformula of the compounds of the present invention having the naphthyridine scaffold,

FIG. 2 shows representative examples of the inventive naphthyridine-derived compounds,

FIG. 3 shows the CD spectrum of the [1,6]naphthyridine-2-carboxylic acid ((R)-1-phenyl-ethyl)-amide and [1,6]naphthyridine-2-carboxylic acid ((S)-1-phenyl-ethyl)-amide.

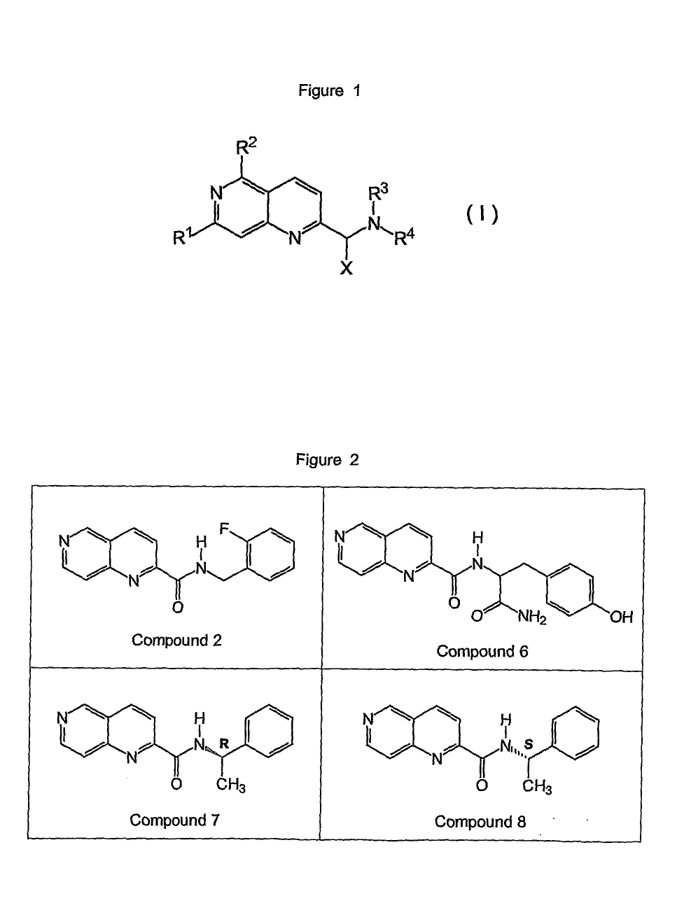

### EXPERIMENTAL PART

### Synthesis of Compounds

The naphthyridine-derived compounds can be prepared according to the ways as disclosed in WO 97/34894 A1 and WO 99/29318 A1.

Below three general methods are described:

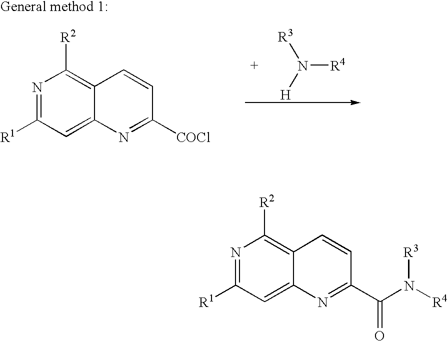

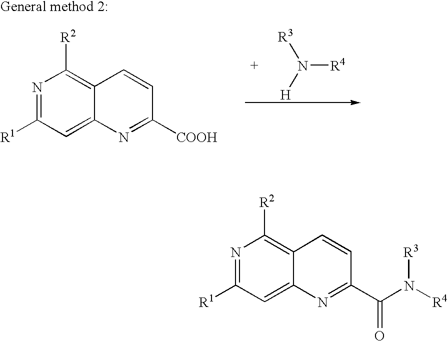

14.00 mmol of corresponding amino derivative and 10.00 mmol of carboxylic acid, 11,00 mmol of 1-hydroxybenzotriazole and 11.10 mmol of N′-(3-dimethylaminopropyl)-N-ethylcarbodiimide hydrochloride in 120 cm³ N,N dimethylformamide was stirred overnight at room temperature. Then 1000 g of crashed ice was added and stirred for one hour. The precipiate was filtered off, washed with saturated NaHCO₃ solution, water and dried at room temperature. The crude material was refluxed in ethylalcohol for 10 minutes, cooled back and filtered off. Yield 40-60%

The following compounds were prepared by this method:
[1,6]Naphthyridine-2-carboxylic acid (3-chloro-phenyl)-amide, [1,6]Naphthyridine-2-carboxylic acid (3-bromo-phenyl)-amide, [1,6]Naphthyridine-2-carboxylic acid indan-1-ylamide, [1,6]Naphthyridine-2-carboxylic acid 2-trifluoromethyl-benzylamide, [1,6]Naphthyridine-2-carboxylic acid cyclohexylmethyl-amide, [1,6]Naphthyridine-2-carboxylic acid 2-chloro-benzylamide, [1,6]Naphthyridine-2-carboxylic acid 3-bromo-benzylamide, [1,6]Naphthyridine-2-carboxylic acid (1,2,3,4-tetrahydro-naphthalen-1-yl)-amide, [1,6]Naphthyridine-2-carboxylic acid 2-amino-6-fluoro-benzylamide, [1,6]Naphthyridine-2-carboxylic acid (3-morpholin-4-yl-propyl)-amide, [1,6]Naphthyridine-2-carboxylic acid 3-methyl-benzylamide, [1,6]Naphthyridine-2-carboxylic acid [3-(4-methyl-piperazin-1-yl)-propyl]-amide, [1,6]Naphthyridine-2-carboxylic acid phenethyl-amide, [1,6]Naphthyridine-2-carboxylic acid 2-fluoro-benzylamide, [1,6]Naphthyridine-2-carboxylic acid 4-chloro-benzylamide, [1,6]Naphthyridine-2-carboxylic acid 4-bromo-benzylamide, [1,6]Naphthyridine-2-carboxylic acid 2-ethoxy-benzylamide, [1,6]Naphthyridine-2-carboxylic acid (2-phenyl-cyclopropyl)-amide, [1,6]Naphthyridine-2-carboxylic acid (1-phenyl-ethyl)-amide, (3,4-Dihydro-1H-isoquinolin-2-yl)-[1,6]naphthyridin-2-yl-methanone, [1,6]Naphthyridine-2-carboxylic acid 2-methoxy-benzylamide, [1,6]Naphthyridine-2-carboxylic acid [2-(1H-indol-3-yl)-ethyl]-amide, [1,6]Naphthyridine-2-carboxylic acid 4-fluoro-benzylamide, [1,6]Naphthyridine-2-carboxylic acid 3,4-dimethoxy-benzylamide, [1,6]Naphthyridine-2-carboxylic acid 2-methyl-benzylamide, [1,6]Naphthyridine-2-carboxylic acid 3-trifluoromethyl-benzylamide, [1,6]Naphthyridine-2-carboxylic acid cycloheptylamide, [1,6]Naphthyridine-2-carboxylic acid 4-trifluoromethyl-benzylamide, [1,6]Naphthyridine-2-carboxylic acid (3-imidazol-1-yl-propyl)-amide, [1,6]Naphthyridine-2-carboxylic acid [2-(isoquinoline-5-sulfonylamino)-ethyl]-amide, [1,6]Naphthyridine-2-carboxylic acid 4-methyl-benzylamide, [1,6]Naphthyridine-2-carboxylic acid 3-chloro-benzylamide, [1,6]Naphthyridine-2-carboxylic acid 4-methoxy-benzylamide, [4-(Isoquinoline-5-sulfonyl)-piperazin-1-yl]-[1,6]naphthyridin-2-yl-methanone, [1,6]Naphthyridine-2-carboxylic acid (benzo[1,3]dioxol-5-ylmethyl)-amide, [1,6]Naphthyridine-2-carboxylic acid [3-(2-oxo-pyrrolidin-1-yl)-propyl]-amide, [1,6]Naphthyridine-2-carboxylic acid 2-bromo-benzylamide, [1,6]Naphthyridine-2-carboxylic acid (2,4-dimethyl-phenyl)-amide, [1,6]Naphthyridine-2-carboxylic acid (4-chloro-2-methyl-phenyl)-amide, [1,6]Naphthyridine-2-carboxylic acid (3,5-bis-trifluoromethyl-phenyl)-amide, [1,6]Naphthyridine-2-carboxylic acid (2-fluoro-phenyl)-amide, [1,6]Naphthyridine-2-carboxylic acid (1H-[1,2,4]triazol-3-yl)-amide, [1,6]Naphthyridine-2-carboxylic acid (4-fluoro-phenyl)-amide, [1,6]Naphthyridine-2-carboxylic acid (3-ethyl-phenyl)-amide, [1,6]Naphthyridine-2-carboxylic acid (3,5-diacetyl-phenyl)-amide, [1,6]Naphthyridine-2-carboxylic acid benzylamide, [1,6]Naphthyridine-2-carboxylic acid [3-(4-pyridin-3-yl-pyrimidin-2-ylamino)-phenyl]-amide, [1,6]Naphthyridine-2-carbothioic acid [3-(4-pyridin-3-yl-pyrimidin-2-ylamino)-phenyl]-amide, [1,6]Naphthyridine-2-carboxylic acid 3-methoxy-benzylamide, [1,6]Naphthyridine-2-carboxylic acid (1-carbamoyl-2-phenyl-ethyl)-amide, [1,6]Naphthyridine-2-carboxylic acid [1-carbamoyl-2-(4-hydroxy-phenyl)-ethyl]-amide, [1,6]Naphthyridine-2-carboxylic acid 1-phenyl-propylamide, [1,6]Naphthyridine-2-carboxylic acid 1-(4-fluoro-phenyl)-ethylamide, [1,6]Naphthyridine-2-carboxylic acid pyrid-3-yl-methylamide, [1,6]Naphthyridine-2-carboxylic acid pyrid-4-yl-methylamide, [1,6]Naphthyridine-2-carboxylic acid thiophen-2-yl-methylamide, [1,6]Naphthyridine-2-carboxylic acid 1,2-diphenyl-ethylamide, [1,6]Naphthyridine-2-carboxylic acid 2-(4-chloro-phenyl)-ethylamide, [1,6]Naphthyridine-2-carboxylic acid 1-(4-bromo-phenyl)-ethylamide, [1,6]Naphthyridine-2-carboxylic acid 2-(4-bromo-phenyl)-ethylamide, [1,6]Naphthyridine-2-carboxylic acid pyrid-2-yl-methylamide, [1,6]Naphthyridine-2-carboxylic acid diphenyl-methylamide, [1,6]Naphthyridine-2-carboxylic acid 1-(4-chlorophenyl)-propylamide, [1,6]Naphthyridine-2-carboxylic acid 1-(4-methylphenyl)-ethylamide, [1,6]Naphthyridine-2-carboxylic acid 1-(4-chlorophenyl)-ethylamide, [1,6]Naphthyridine-2-carboxylic acid 1-(4-methoxyphenyl)-ethylamide, [1,6]Naphthyridine-2-carboxylic add 1-(3-chlorophenyl)-propylamide, [1,6]Naphthyridine-2-carboxylic acid 1-(4-fluorophenyl)-ethylamide, [1,6]Naphthyridine-2-carboxylic acid 1-(4-methylphenyl)-propylamide, [1,6]Naphthyridine-2-carboxylic acid 1-(thiophen-2-yl)-propylamide, [1,6]Naphthyridine-2-carboxylic acid 1-(thiophen-3-yl)-ethylamide, [1,6]Naphthyridine-2-carboxylic acid N-methyl-N-(1-phenyl ethyl)-amide.

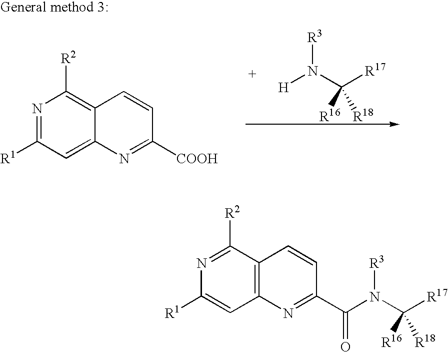

3 mmol 1,6-naphthyridine carboxylic acid, 3.3 mmol 1-hydroxybenzotriazole, 4.5 mmol of a chiral compound such as R-(+)-1-phenylethylamine and 3.3 mmol N′-(3-dimethylaminopropyl)-N-ethylcarbodiimide hydrochloride was stirred in 100 ml abs. DMF overnight at room temperature. 400 ml cold water was added and stirred for additional 1 hour. Extracted with ethyl acetate, washed with NaHCO₃, saturated NaCl, dried over MgSO₄. The solvent was removed, the yellow oil was chromatographed on silica gel eluted with ethylacetate. Yield about 45%

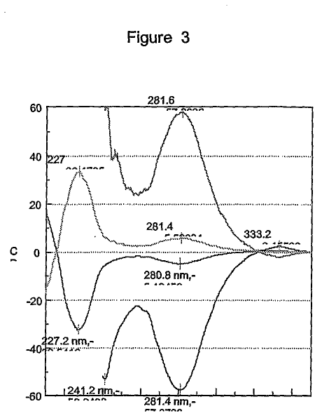

The optical purity of enantiomers was studied with CD spectroscopy. The CD spectrum of the [1,6]naphthyridine-2-carboxylic acid ((R)-1-phenyl-ethyl)-amide and [1,6]naphthyridine-2-carboxylic acid ((S)-1-phenyl-ethyl)-amide is shown in FIG. 3. It can be seen that these molecules are enantiomers and have the same optically purity.

The following compounds were prepared by this method:
[1,6]Naphthyridine-2-carboxylic acid ((R)-1-phenyl-ethyl)-amide,
[1,6]Naphthyridine-2-carboxylic acid ((S)-1-phenyl-ethyl)-amide,
[1,6]Naphthyridine-2-carboxylic acid ((R)-1-carbamoyl-2-phenyl-ethyl)-amide,
[1,6]Naphthyridine-2-carboxylic acid ((S)-1-carbamoyl-2-phenyl-ethyl)-amide,
[1,6]Naphthyridine-2-carboxylic acid [(R)-1-carbamoyl-2-(4-hydroxy-phenyl)-ethyl]-amide,
[1,6]Naphthyridine-2-carboxylic acid [(S)-1-carbamoyl-2-(4-hydroxy-phenyl)-ethyl]-amide,
[1,6]Naphthyridine-2-carboxylic acid (S) (1-carbamoyl-ethyl)-amide,
[1,6]Naphthyridine-2-carboxylic acid (R) (1-carbamoyl-ethyl)-amide,
[1,6]Naphthyridine-2-carboxylic acid ((R)-1-carbamoyl-2-methyl-propyl)-amide,
[1,6]Naphthyridine-2-carboxylic acid ((S)-1-carbamoyl-2-methyl-propyl)-amide,
[1,6]Naphthyridine-2-carboxylic acid ((R)-1-carbamoyl-3-methyl-butyl)-amide,
[1,6]Naphthyridine-2-carboxylic acid ((S)-1-carbamoyl-3-methyl-butyl)-amide,
[1,6]Naphthyridine-2-carboxylic acid ((R)-1-carbamoyl-3-methylsulfanyl-propyl)-amide,
[1,6]Naphthyridine-2-carboxylic acid ((S)-1-carbamoyl-3-methylsulfanyl-propyl)-amide,
[1,6]Naphthyridine-2-carboxylic acid [(S)-1-carbamoyl-2-(1H-indol-3-yl)-ethyl]-amide,
[1,6]Naphthyridine-2-carboxylic acid [(R)-1-carbamoyl-2-(1H-indol-3-yl)-ethyl]-amide,
[1,6]Naphthyridine-2-carboxylic acid [(S)-1-carbamoyl-2-(1H-imidazol-4-yl)-ethyl]-amide,
[1,6]Naphthyridine-2-carboxylic acid [(R)-1-carbamoyl-2-(1H-imidazol-4-yl)-ethyl]-amide,
(R)-[1,6]Naphthyridine-2-carboxylic acid 1-(naphth-2-yl)-ethylamide,
(S)-[1,6]Naphthyridine-2-carboxylic acid 1-(naphth-2-yl)-ethylamide,
(R)-[1,6]Naphthyridine-2-carboxylic acid 1-(4-methylphenyl)-ethylamide,
(R)-[1,6]Naphthyridine-2-carboxylic acid 1-phenyl-propylamide,
(S)-[1,6]Naphthyridine-2-carboxylic acid 1-phenyl-propylamide,
(R)-[1,6]Naphthyridine-2-carboxylic acid 1-(4-chloro-phenyl)-ethylamide,
(S)-[1,6]Naphthyridine-2-carboxylic acid 1-(4-methoxy-phenyl)-ethylamide.

### Materials and Methods

### Cellular Assays:

### Phosphorylation of MBS (Myosin Binding Subunit) of MLC Phosphatase by ROCK

HEK293 cells were grown in DMEM containing 10% calf serum, 1 mM sodium pyruvate, penicillin and streptomycin at 37° C. in the presence of 5% CO₂. 5.0×10⁵ cells were seeded in six-well dishes (Greiner). Cells were transfected with 500 ng RhoA, or RhoA-GV14, or ROCK1 (full length or kinase domain), or ROCK2 (full length or kinase domain), which were cloned in pcDNA3 or pPM7 (expression driven by CMV promoters), in a volume of 100 μl in the presence of 0.125 mM CaCl₂ and 1×BBS, pH 6.85. Cells were harvested 16 h post transfection. Alternatively, cells were serum deprived for 24 h, then stimulated with 20 μM LPA (lysophoshatidic acid) or 200 nM TPA for 15 min.

In experiments with inhibitors, cells were treated for 30 min post transfection with compounds in various concentrations before the addition of the stimuli.

Cells were lysed in 180 μl of lysis buffer (20 mM Tris/HCl, pH 7.5; 150 mM NaCl, 10% glycerol, 1 mM EDTA, pH8.0; 1% Triton X-100, 10 μg/ml aprotinin, 10 μg leupeptin, 1 mM PMSF, 30 mM NaF, 1 mM Na₃VO₄, and DNase I (10 μg/ml). Lysates were either directly used for SDS PAGE (30 μg protein/sample), and MBS phosphorylation at Thr-850 was detected with an anti-phospho MBS-specific rabbit polyclonal IgG (#36-003, Upstate), or immunoprecipitated with 5 μl anti-MBS (Upstate #36-003, or #07-159) and 40 μl protein G sepharose for 16 h at 4° C. After washing with 750 μl (1% Triton X-100 in 1×PBS) the immunoprecipitates were separated by SDS PAGE, and blotted onto nitrocellulose membranes. Detection was performed with an anti-MBS antibody (#07-251, Upstate), followed by a polyclonal goat anti-rabbit IgG coupled to HRP (P0448, DAKO). The ECL chemiluminescence kit (Amersham) was used for visualization.

### SRF Luciferase Reporter Gene Assay:

HEK293 cells were grown in DMEM supplemented with 10% fetal calf serum, 1 mM sodium pyruvate, penicillin and streptomycin at 37° C. in the presence of 5% CO₂. Transient transfections were performed according to the calcium phosphate method (see above).

Cells were transfected with pSRF-Luc (Stratagene) and vectors like pcDNA3, or pPM7, expressing RhoA, or RhoA-GV14, or ROCK1 (full length or kinase domain), or ROCK2 (full length or kinase domain), under the control of the CMV promoter. 24 h after serum starvation cells were stimulated with fetal calf serum (15%), LPA (10 μM), or sphingosine (10 μM). For testing compound efficiency compounds were added 30 minutes before stimulation. After 8 h of incubation cells were lysed in 25 mM Tris/HCl, pH 7.5, 2 mM DTT, 1% Triton X-100, 10% glycerol. Light emission was quantitated with the multimode reader “Analyst GT (Molecular Devices). Inhibition of luciferase activity by the compounds was calculated as percentage of the activity elicited by the stimulus.

### Cell Viability Assay

The human carcinoma cell lines A2780 (ovarian), HCT116 (colon), U373 (glioblastoma), PM1 (lymphoid) and the mouse macrophage cell line J774 were grown in 384 well plates (100-250 cells per well in a volume of 25 μl) in either RPMI 1640 media (A2780, HCT116, PM1) or DMEM (U373, J774) supplemented with 10% fetal calf serum, and 2 mM glutamine. After 24 h cells were treated with compounds and grown for further 3 days. 5 μl Alamar Blue™ dye (#DAL1025, Biosource Inc.) was added to the cells and 4 h later cell viability was monitored with the multimode reader “Analyst GT (Molecular Devices) measuring at 560 and 590 nm.

### Determination of ROCK Kinase Activity

IC₅₀ values for the various compounds were determined with recombinant human ROCK1 (amino acids 1-555, aminoterminal 6×His tag) and recombinant human ROCK2 (amino acids 1-530, aminoterminal 6×His tag) under the following reaction conditions:

### Kinase: ROCK1 Human

IMAP Assay

| | |
|---|---|
| Reaction Volume: | 8 μl |
| Reaction Time: | 60 min |
| Reaction Temperature: | room temperature |
| IMAP Incubation Time: | 60 min |
| Assay Plate: | 384 well U bottom, PP, black, |
| | low volume (Corning, 3676) |
| Compound Plate: | 384 well U bottom, PS (Falcon, 3995) |
| IMAP Binding Buffer A: | Molecular Devices, R7282 |
| IMAP Binding Buffer B: | Molecular Devices, R7283 |
| IMAP Binding Reagent: | Molecular Devices, R7207 |

### Controls:

| | |
|---|---|
| Negative Control (C−): | no Kinase, no Inhibitor |
| Positive Control (C+): | no Inhibitor |

### Reaction Buffer:

20 mM Hepes, pH 7

0.4 mM MgCl₂

20 mM NaCl

1 mM DTT

0.01% Triton X-100

### Final Assay Concentrations:

| | |
|---|---|
| Kinase: | Kinase conc. yielding |
| | 50% Substr. turn over |
| ATP: | 38.9 μM |
| Substrate: 5FI-AKRRRLSSLRA-NH2 | 400 nM (Jerini) |

| | |
|---|---|
| IMAP Binding Solution: | 100% IMAP Binding Buffer A |
| | 0% IMAP Binding Buffer B |
| | IMAP Binding Reagent 1:400 |

### Pipetting Sequence:

1) Add 6 μl 8/6 fold concentrated Substrate+8/6 fold concentrated ATP in 1 fold concentrated Reaction Buffer to each well of Assay Plate
2) Add 10.8 nl 740 fold concentrated Inhibitor in 100% DMSO to each well except to C− and C+ wells using pintool
3) Add 10.8 nl 100% DMSO to C− and C+ wells using pintool
4) Add 2 μl Reaction Buffer to C− wells
5) Add 2 μl 4 fold concentrated Kinase in Reaction Buffer to each well except C− wells
6) Incubate according to Reaction Time at room temperature
7) Add 15 μl IMAP Binding Solution to each well
8) Incubate according to IMAP Incubation Time at room temperature
9) Measure Fluorescence Polarization
10) Measure Fluorescence Intensity

### Kinase: ROCK2 Human

IMAP Assay

| | |
|---|---|
| Reaction Volume: | 8 μl |
| Reaction Time: | 60 min |
| Reaction Temperature: | room temperature |
| IMAP Incubation Time: | 60 min |
| Assay Plate: | 384 well U bottom, PP, black, |
| | low volume (Corning, 3676) |
| Compound Plate: | 384 well U bottom, PS (Falcon, 3995) |
| IMAP Binding Buffer A: | Molecular Devices, R7282 |
| IMAP Binding Buffer B: | Molecular Devices, R7283 |
| IMAP Binding Reagent: | Molecular Devices, R7207 |

### Controls:

| | |
|---|---|
| Negative Control (C−): | no Kinase, no Inhibitor |
| Positive Control (C+): | no Inhibitor |

### Reaction Buffer:

20 mM Mops, pH 7.5

0.6 mM MgCl₂

1 mM DTT

0.01% Tween20

### Final Assay Concentrations:

| | |
|---|---|
| Kinase: | Kinase conc. yielding |
| | 50% Substr. turn over |
| ATP: | 34.8 μM |
| Substrate: 5FI-AKRRRLSSLRA-NH2 | 400 nM (Jerini) |

| | |
|---|---|
| IMAP Binding Solution: | 100% IMAP Binding Buffer A |
| | 0% IMAP Binding Buffer B |
| | IMAP Binding Reagent 1:400 |

### Pipetting Sequence:

11) Add 6 μl 8/6 fold concentrated Substrate+8/6 fold concentrated ATP in 1 fold concentrated Reaction Buffer to each well of Assay Plate
12) Add 10.8 nl 740 fold concentrated Inhibitor in 100% DMSO to each well except to C− and C+ wells using pintool
13) Add 10.8 nl 100% DMSO to C− and C+ wells using pintool
14) Add 2 μl Reaction Buffer to C− wells
15) Add 2 μl 4 fold concentrated Kinase in Reaction Buffer to each well except C− wells
16) Incubate according to Reaction Time at room temperature
17) Add 15 μl IMAP Binding Solution to each well
18) Incubate according to IMAP Incubation Time at room temperature
19) Measure Fluorescence Polarization
20) Measure Fluorescence Intensity

IC₅₀ determination of [1,6]Naphthyridines on ROCK1 and 2

Table 2 shows the IC₅₀ values of [1,6]Naphthyridines determined by in vitro kinase assays on ROCK1 and 2.

TABLE 2

| | IC50 | IC50 |
|---|---|---|
| | ROCK2 | Rock1 |
| Compound | [μM] | [μM] |
| [1,6]Naphthyridine-2-carboxylic acid 2-chloro- | 3.83 | 4.77 |
| benzylamide | | |
| [1,6]Naphthyridine-2-carboxylic acid 3-bromo- | 1.04 | 0.91 |
| benzylamide | | |
| [1,6]Naphthyridine-2-carboxylic acid 2-fluoro- | 2.08 | 0.79 |
| benzylamide | | |
| [1,6]Naphthyridine-2-carboxylic acid 4-chloro- | 2.11 | 2.05 |
| benzylamide | | |
| [1,6]Naphthyridine-2-carboxylic acid 4-bromo- | 1.33 | 1.37 |
| benzylamide | | |
| [1,6]Naphthyridine-2-carboxylic acid 2-ethoxy- | 13.01 | n.d. |
| benzylamide | | |
| [1,6]Naphthyridine-2-carboxylic acid (1-phenyl- | 1.59 | 0.71 |
| ethyl)-amide | | |
| [1,6]Naphthyridine-2-carboxylic acid 4-methyl- | 2.23 | 1.28 |
| benzylamide | | |
| [1,6]Naphthyridine-2-carboxylic acid 3-chloro- | 2.57 | 0.61 |
| benzylamide | | |
| [1,6]Naphthyridine-2-carboxylic acid 4-methoxy- | 5.12 | 6.04 |
| benzylamide | | |
| [1,6]Naphthyridine-2-carboxylic acid | 1.87 | 1.46 |
| (benzo[1,3]dioxol-5-ylmethyl)-amide | | |
| [1,6]Naphthyridine-2-carboxylic acid 2-bromo- | 7.23 | 2.24 |
| benzylamide | | |
| [1,6]Naphthyridine-2-carboxylic acid benzylamide | 7.68 | 3.15 |
| [1,6]Naphthyridine-2-carboxylic acid 3-methoxy- | 3.10 | 3.34 |
| benzylamide | | |
| (S)-[1,6]Naphthyridine-2-carboxylic acid (1- | 5.64 | 8.16 |
| carbamoyl-2-phenyl-ethyl)-amide | | |
| (S)-[1,6]Naphthyridine-2-carboxylic acid [1- | 4.26 | 6.83 |
| carbamoyl-2-(4-hydroxy-phenyl)-ethyl]-amide | | |
| [1,6]Naphthyridine-2-carboxylic acid ((R)-1-phenyl- | 0.92 | 1.13 |
| ethyl)-amide | | |
| [1,6]Naphthyridine-2-carboxylic acid 1-phenyl- | 0.50 | 0.66 |
| propylamide | | |
| (R)-[1,6]Naphthyridine-2-carboxylic acid 1-(naphth- | 0.88 | 2.32 |
| 2-yl)-ethylamide | | |
| [1,6]Naphthyridine-2-carboxylic acid thiophen-2-yl- | 7.84 | 1.97 |
| methylamide | | |
| [1,6]Naphthyridine-2-carboxylic acid 2-(4-chloro- | 5.70 | 1.91 |
| phenyl)-ethylamide | | |
| [1,6]Naphthyridine-2-carboxylic acid 1-(4-bromo- | 0.89 | 0.97 |
| phenyl)-ethylamide | | |
| [1,6]Naphthyridine-2-carboxylic acid 2-(4-bromo- | 1.52 | 1.50 |
| phenyl)-ethylamide | | |
| [1,6]Naphthyridine-2-carboxylic acid 1-(4- | 0.48 | 1.26 |
| chlorophenyl)-propylamide | | |
| [1,6]Naphthyridine-2-carboxylic acid 1-(4- | 1.41 | 1.29 |
| methylphenyl)-ethylamide | | |
| [1,6]Naphthyridine-2-carboxylic acid 1-(4- | 0.77 | 1.10 |
| chlorophenyl)-ethylamide | | |
| [1,6]Naphthyridine-2-carboxylic acid 1-(4- | 10.74 | n.d. |
| methoxyphenyl)-ethylamide | | |
| [1,6]Naphthyridine-2-carboxylic acid 1-(3- | 0.38 | 0.30 |
| chlorophenyl)-propylamide | | |
| [1,6]Naphthyridine-2-carboxylic acid 1-(4- | 0.34 | 0.94 |
| fluorophenyl)-ethylamide | | |
| [1,6]Naphthyridine-2-carboxylic acid 1-(4- | 1.26 | 0.79 |
| methylphenyl)-propylamide | | |
| [1,6]Naphthyridine-2-carboxylic acid 1-(thiophen-2- | 1.01 | n.d. |
| yl)-propylamide | | |
| [1,6]Naphthyridine-2-carboxylic acid 1-(thiophen-3- | 1.91 | n.d. |
| yl)-ethylamide | | |
| (R)-[1,6]Naphthyridine-2-carboxylic acid 1-(4- | 0.41 | n.d. |
| methylphenyl)-ethylamide | | |
| (R)-[1,6]Naphthyridine-2-carboxylic acid 1-phenyl- | 0.16 | n.d. |
| propylamide | | |
| (R)-[1,6]Naphthyridine-2-carboxylic acid 1-(4-chloro- | 0.36 | n.d. |
| phenyl)-ethylamide | | |

1. Compounds having the general formula (I): 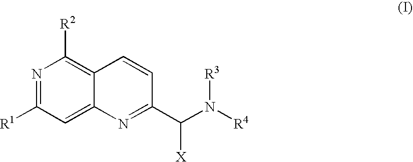
wherein
R¹, R², R²⁶-R⁶⁹ represent independently of each other —H, —OH, —OCH₃, —OC₂H₅, —OC₃H₇, —O-cyclo-C₃H₅, —OCH(CH₃)₂, —OC(CH₃)₃, —OC₄H₉, —OPh, —OCH₂-Ph, —OCPh₃, —SH, —SCH₃, —SC₂H₅, —SC₃H₇, —S-cyclo-C₃H₅, —SCH(CH₃)₂, —SC(CH₃)₃, —NO₂, —F, —Cl, —Br, —I, —N₃, —CN, —OCN, —NCO, —SCN, —NCS, —CHO, —COCH₃, —OC₂H₅, —COC₃H₇, CO-cyclo-C₃H₅, —COCH(CH₃)₂, —COC(CH₃)₃, —OOH, —COCN, —COOCH₃, —COOC₂H₅, —COOC₃H₇, —COO-Cyclo-C₃H₅, —COOCH(CH₃)₂, —COOC(CH₃)₃, —OOC—CH₃, —OOC—C₂H₅, —OOC—C₃H₇, —OOC-Cyclo-C₃H₅, —OOC—CH(CH₃)₂, —OOC—C(CH₃)₃, —CONH₂, —CONHCH₃, —CONHC₂H₅, —CONHC₃H₇, —CONH-cyclo-C₃H₅, —CONH[CH(CH₃)₂], —CONH[C(CH₃)₃], —CON(CH₃)₂, —CON(C₂H₅)₂, —CON(C₃H₇)₂, —CON(cyclo-C₃H₅)₂, —CON[CH(CH₃)₂]₂, —CON[C(CH₃)₃]₂, —NH₂, —NHCH₃, —NHC₂H₅, —NHC₃H₇, —NH-cyclo-C₃H₅, —NHCH(CH₃)₂, —NHC(CH₃)₃, —N(CH₃)₂, —N(C₂H₅)₂, —N(C₃H₇)₂, —N(cyclo-C₃H₅)₂, —N[CH(CH₃)₂]₂, —N[C(CH₃)₃]₂, —SOCH₃, —SOC₂H₅, —SOC₃H₇, —SO-cyclo-C₃H₅, —SOCH(CH₃)₂, —SOC(CH₃)₃, —SO₂CH₃, —SO₂C₂H₅, —SO₂C₃H₇, —SO₂-cyclo-C₃H₅, —SO₂CH(CH₃)₂, —SO₂C(CH₃)₃, —SO₃H, —SO₃CH₃, —SO₃C₂H₅, —SO₃C₃H₇, —SO₃-cyclo-C₃H₅, —SO₃CH(CH₃)₂, —SO₃C(CH₃)₃, —OCF₃, —OC₂F₅, —O—COOCH₃, —O—COOC₂H₅, —O—COOC₃H₇, —O—COO-cyclo-C₃H₅, —O—COOCH(CH₃)₂, —O—COOC(CH₃)₃, —NH—CO—NH₂, —NH—CO—NHCH₃, —NH—CO—NHC₂H₅, —NH—CO—NHC₃H₇, —NH—CO—NH-cyclo-C₃H₅, —NH—CO—NH[CH(CH₃)₂], —NH—CO—NH[C(CH₃)₃], —NH—CO—N(CH₃)₂, —NH—CO—N(C₂H₅)₂, —NH—CO—N(C₃H₇)₂, —NH—CO—N(cyclo-C₃H₅)₂, —NH—CO—N[CH(CH₃)₂]₂, —NH—CO—N[C(CH₃)₃]₂, —NH—CS—NH₂, —NH—CS—NH-cyclo-C₃H₅, —NH—CS—NHC₃H₇, —NH—CS—NH[CH(CH₃)₂], —NH—CS—NH[C(CH₃)₃], —NH—CS—N(CH₃)₂, —NH—CS—N(C₂H₅)₂, —NH—CS—N(C₃H₇)₂, —NH—CS—N(cyclo-C₃H₅)₂, —NH—C(═NH)—NH₂, —NH—CS—NHC₂H₅, —NH—CS—NHCH₃, —OCO—NH₂, —O—CONHCH₃, —O—CONHC₂H₅, —O—CO—NHC₃H₇, —O—CO—NH-cyclo-C₃H₅, —O—CON—NH[CH(CH₃)₂], —O—CO—NH[C(CH₃)₃], —O—CO—N(CH₃)₂, —O—CO—N(C₂H₅)₂, —CO—N(C₃H₇)₂, —O—CO—N (cyclo-C₃H₅)₂, —O—CON[CH(CH₃)₂]₂, —O—CON[C(CH₃)₃]₂, —O—CO—OCH₃, —O—CO—OC₂H₅, —O—CO—C₃H₇, —O—CO—O-cyclo-C₃H₅, —O—CO—OCH(CH₃)₂, —O—CO—OC(CH₃)₃, —CH₂F —CHF₂, —CF₃, —CH₂Cl, —CHCl₂, —CCl₃, —CH₂Br —CHBr₂, —CBr₃, —CPh₃, —CH₂—CH₂F —CH₂—CHF₂, —CH₂—CF₃, —CH₂—CH₂Cl, —CH₂—CHCl₂, —CH₂—CCl₃, —CH₂—CH₂Br —CH₂—CHBr₂, —CH₂—CBr₃, —CH₃, —C₂H₅, —C₃H₇, -cyclo-C₃H₅, —CH(CH₃)₂, —C(CH₃)₃, —C₄H₉, —CH₂—CH(CH₃)₂, —CH(CH₃)—C₂H₅, —C(CH₃)₃, —C₅H₁₁, —C₆H₁₃, —C₇H₁₅, —C₈H₁₇, —C₉H₁₉, —C₁₀H₂₁, -Ph, —CH₂-Ph, —CH═CH₂, —CH₂—CH═CH₂, —C(CH₃)═CH₂, —CH═CH—CH₃, —C₂H₄—CH═CH₂, —CH═C(CH₃)₂, —C≡CH, —C≡C—CH₃, —CH₂—C≡CH;
R³, R¹¹ represent independently of each other —H, —CH₃, —C₂H₅, —C₃H₇, -cyclo-C₃H₅, —CH(CH₃)₂, —C(CH₃)₃, —C₄H₉, —CH₂—CH(CH₃)₂, —CH(CH₃)—C₂H₅, —C(CH₃)₃, —C₅H₁₁, —C₆H₁₃, —C₇H₁₅, —C₈H₁₇, —C₉H₁₉, —C₁₀H₂₁, -Ph, —CH₂-Ph, —CH═CH₂, —CH₂—CH═CH₂, —C(CH₃)═CH₂, —CH═CH—CH₃, —C₂H₄—CH═CH₂, —CH═C(CH₃)₂, —C≡CH, —C≡C—CH₃, —CH₂—C≡CH;
the moiety 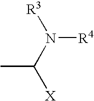
represents 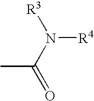
or R*;
R⁴ represents R** or 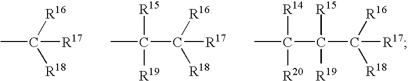
R* and R** represent independently of each other 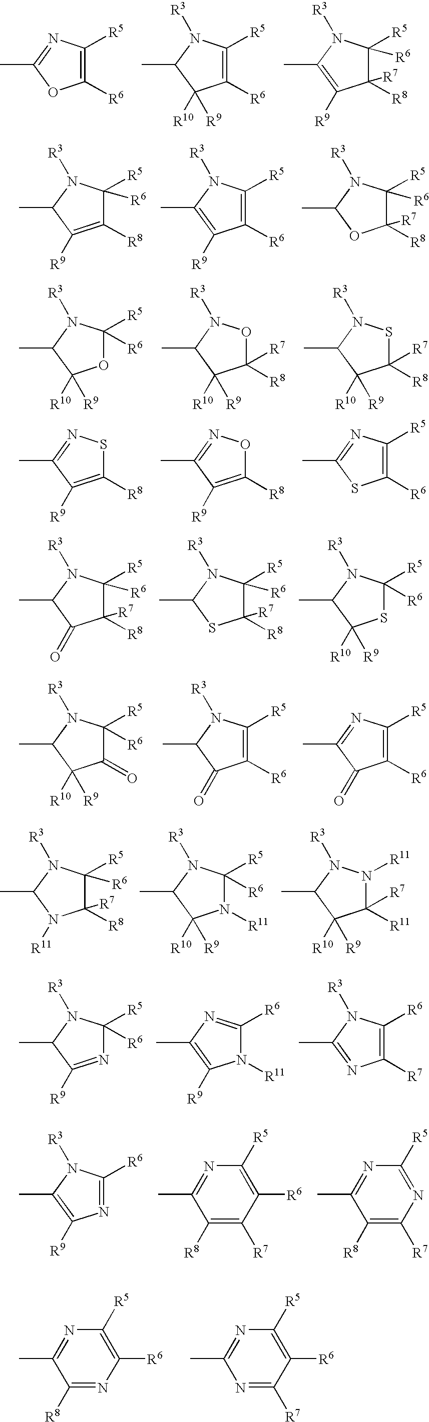 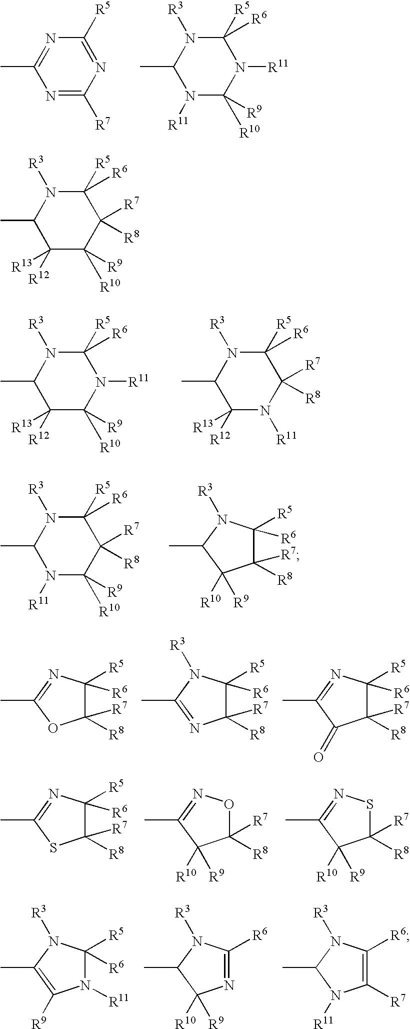
R# represents R** or 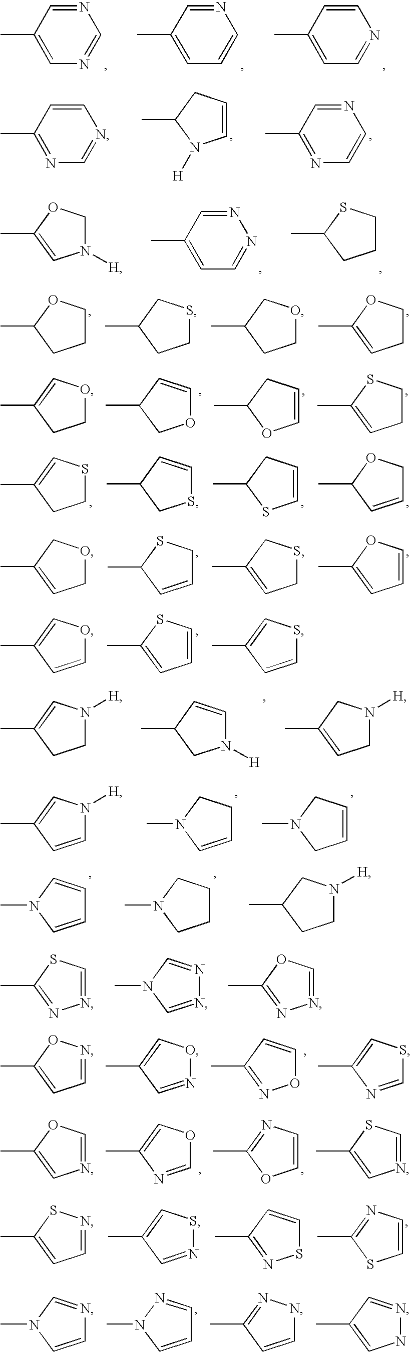 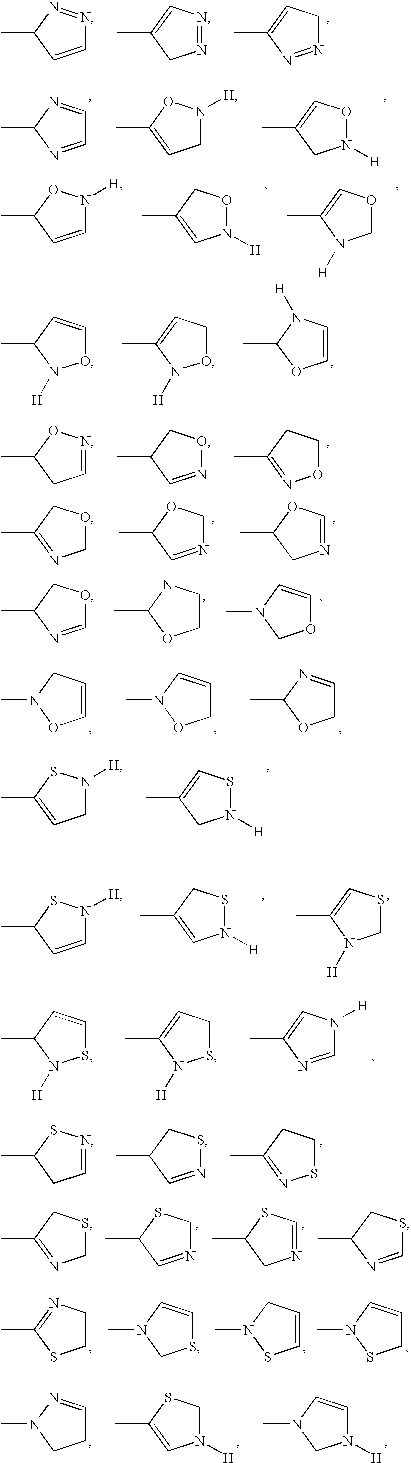 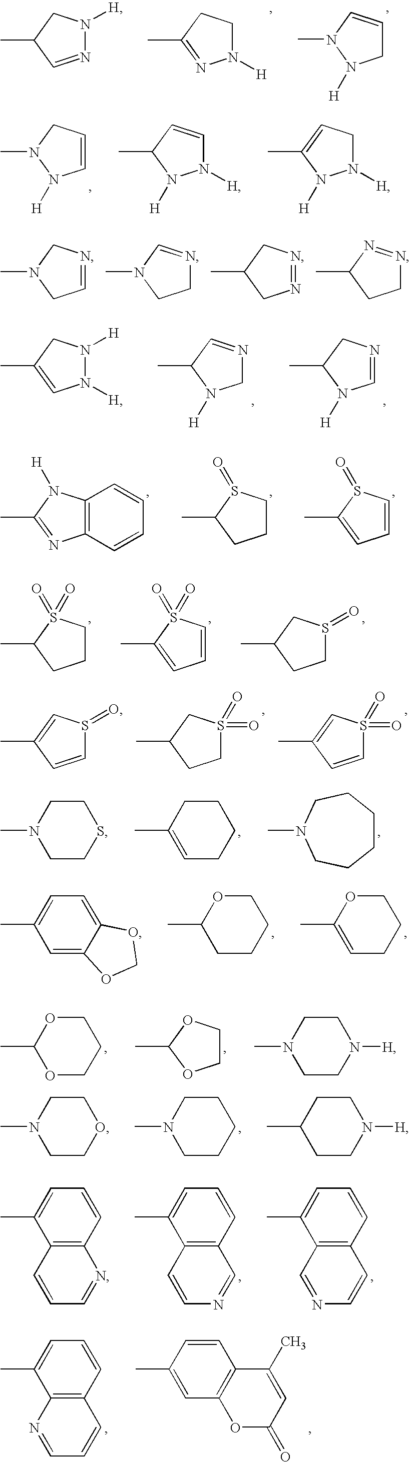 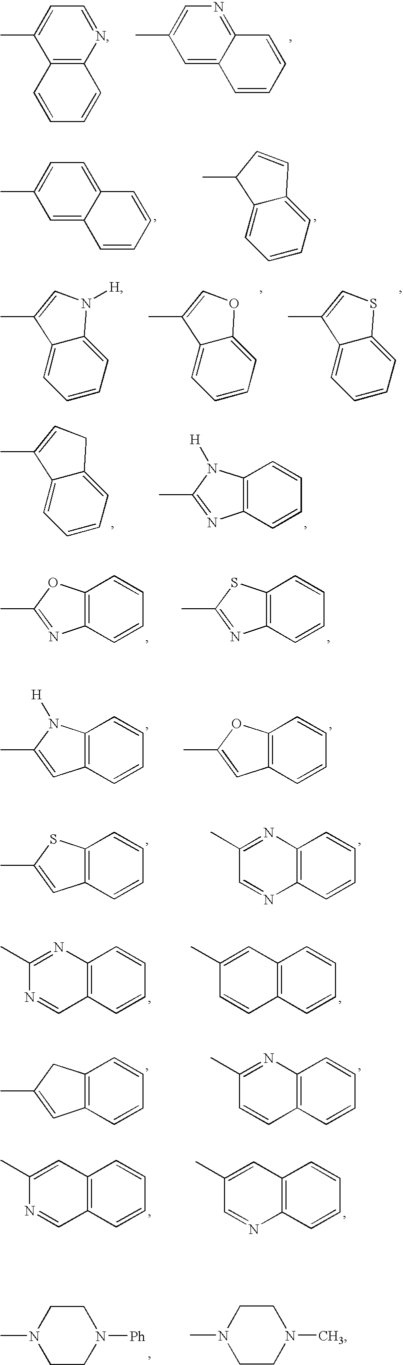
wherein the above-mentioned heteroaromatic and heterocyclic substituents can further bear one, two, three or more substituents selected from R⁵-R¹⁰;
R¹⁵, R¹⁷, R²⁰ represent independently of each other 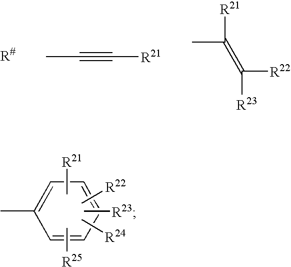
R⁵-R¹⁰, R¹²-R²⁵ represent independently of each other R⁵⁰-R⁶⁹, 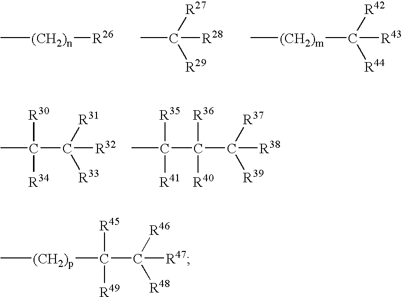
m, n, p are independently of each other integer from 0-10;
and stereoisomeric forms, prodrugs, solvates, hydrates and/or pharmaceutically acceptable salts thereof,
under the proviso that,
if R⁴ represents 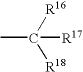
and R¹⁷ represents 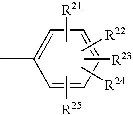
R¹⁶ is different from —H and —CH₃ or at least one of the substituents R²¹-R²⁵ is different from hydrogen.

2. Compound according to claim 1 having the general formula II, 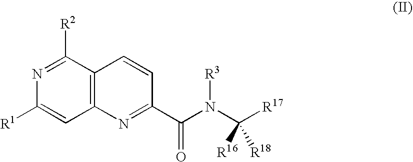
wherein
R¹-R³ and R¹⁶-R¹⁸ have the meanings as defined in claim 1.

3. Compound according to claim 2, wherein
R¹, R², R³ and R¹⁸ are hydrogen;
R¹⁶ represents —OH, —OCH₃, —OC₂H₅, —C₃H₇, —O-cyclo-C₃H₅, —OCH(CH₃)₂, —SH, —SCH₃, —SC₂H₅, —SC₃H₇, —S-cyclo-C₃H₅, —SCH(CH₃)₂, —SC(CH₃)₃, —NO₂, —F, —Cl, —Br, —I, —N₃, —CN, —OCN, —NCO, —SCN, —NCS, —CHO, —COCH₃, —COC₂H₅, —COOH, —COCN, —COOCH₃, —COOC₂H₅, —CONH₂, —CONHCH₃, —CONHC₂H₅, —CON(CH₃)₂, —NH₂, —NHCH₃, —NHC₂H₅, —NHC₃H₇, —NH-cyclo-C₃H₅, —NHCH(CH₃)₂, —SOCH₃, —SOC₂H₅, —SOC₃H₇, —SO-cyclo-C₃H₅, —SOCH(CH₃)₂, —OCF₃, —OC₂F₅, —CH₂F —CHF₂, —CF₃, —CH₂Cl, —CHCl₂, —CCl₃, —CH₂Br —CHBr₂, —CBr₃, —CH₂—CH₂F —CH₂—CHF₂, —CH₂—CF₃, —CH₂—CH₂Cl, —CH₂—CHCl₂, —CH₂—CCl₃, —CH₂—CH₂Br —CH₂—CHBr₂, —CH₂—CBr₃, —CH₃, —C₂H₅, —C₃H₇, -cyclo-C₃H₅, —CH(CH₃)₂, C(CH₃)₃, —C₄H₉, —CH₂—CH(CH₃)₂, —CH(CH₃)—C₂H₅, —C(CH₃)₃, —CH═CH₂, —CH₂—CH═CH₂, —C(CH₃)═CH₂, —CH═CH—CH₃, —C₂H₄—CH═CH₂, —CH═C(CH₃)₂, —C≡CH, —C≡C—CH₃, or —CH₂—C≡CH.

4. Compound according to claim 1 having the general formula III, 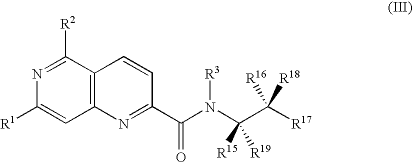
wherein
R¹-R³ and R¹⁵-R¹⁹ have the meanings as defined in claim 1.

5. Compound according to claim 4, wherein
R¹, R², R³ and R¹⁹ are hydrogen;
R¹⁵ represents —OH, —OCH₃, —OC₂H₅, —OC₃H₇, —O-cyclo-C₃H₅, —OCH(CH₃)₂, —SH, —SCH₃, —SC₂H₅, —SC₃H₇, —S-cyclo-C₃H₅, —SCH(CH₃)₂, —SC(CH₃)₃, —NO₂, —F, —Cl, —Br, —I, —N₃, —CN, —OCN, —NCO, —SCN, —NCS, —CHO, —COCH₃, —COC₂H₅, —COOH, —COCN, —COOCH₃, —COOC₂H₅, —CONH₂, —CONHCH₃, —CONHC₂H₅, —CON(CH₃)₂, —NH₂, —NHCH₃, —NHC₂H₅, —NHC₃H₇, —NH-cyclo-C₃H₅, —NHCH(CH₃)₂, —SOCH₃, —SOC₂H₅, —SOC₃H₇, —SO-cyclo-C₃H₅, —SOCH(CH₃)₂, —OCF₃, —OC₂F₅, —CH₂F —CHF₂, —CF₃, —CH₂Cl, —CHCl₂, —CCl₃, —CH₂Br —CHBr₂, —CBr₃, —CH₂—CH₂F —CH₂—CHF₂, —CH₂—CF₃, —CH₂—CH₂CO, —CH₂—CHCl₂, —CH₂—CCl₃, —CH₂—CH₂Br —CH₂—CHBr₂, —CH₂—CBr₃, —CH₃, —C₂H₅, —C₃H₇, -cyclo-C₃H₅, —CH(CH₃)₂, C(CH₃)₃, —C₄H₉, —CH₂—CH(CH₃)₂, —CH(CH₃)—C₂H₅, —C(CH₃)₃, —CH═CH₂, —CH₂—CH═CH₂, —C(CH₃)═CH₂, —CH═CH—CH₃, —C₂H₄—CH═CH₂, —CH═C(CH₃)₂, —C≡CH, —C≡C—CH₃, or —CH₂—C≡CH;

6. Compound according to claim 1, wherein the compound is selected from the group comprising:
[1,6]Naphthyridine-2-carboxylic acid ((R)-1-phenyl-ethyl)-amide,
[1,6]Naphthyridine-2-carboxylic acid ((S)-1-phenyl-ethyl)-amide,
[1,6]Naphthyridine-2-carboxylic acid ((R)-1-carbamoyl-2-phenyl-ethyl)-amide,
[1,6]Naphthyridine-2-carboxylic acid ((S)-1-carbamoyl-2-phenyl-ethyl)-amide,
[1,6]Naphthyridine-2-carboxylic acid [(R)-1-carbamoyl-2-(4-hydroxy-phenyl)-ethyl]-amide,
[1,6]Naphthyridine-2-carboxylic acid [(S)-1-carbamoyl-2-(4-hydroxy-phenyl)-ethyl]-amide,
[1,6]Naphthyridine-2-carboxylic acid (S) (1-carbamoyl-ethyl)-amide,
[1,6]Naphthyridine-2-carboxylic acid (R) (1-carbamoyl-ethyl)-amide,
[1,6]Naphthyridine-2-carboxylic acid ((R)-1-carbamoyl-2-methyl-propyl)-amide,
[1,6]Naphthyridine-2-carboxylic acid ((S)-1-carbamoyl-2-methyl-propyl)-amide,
[1,6]Naphthyridine-2-carboxylic acid ((R)-1-carbamoyl-3-methyl-butyl)-amide,
[1,6]Naphthyridine-2-carboxylic acid ((S)-1-carbamoyl-3-methyl-butyl)-amide,
[1,6]Naphthyridine-2-carboxylic acid ((R)-1-carbamoyl-3-methylsulfanyl-propyl)-amide,
[1,6]Naphthyridine-2-carboxylic acid ((S)-1-carbamoyl-3-methylsulfanyl-propyl)-amide,
[1,6]Naphthyridine-2-carboxylic acid [(S)-1-carbamoyl-2-(1H-indol-3-yl)-ethyl]-amide,
[1,6]Naphthyridine-2-carboxylic acid [(R)-1-carbamoyl-2-(1H-indol-3-yl)-ethyl]-amide,
[1,6]Naphthyridine-2-carboxylic acid [(S)-1-carbamoyl-2-(1H-imidazol-4-yl)-ethyl]-amide,
[1,6]Naphthyridine-2-carboxylic acid [(R)-1-carbamoyl-2-(1H-imidazol-4-yl)-ethyl]-amide.

7. Compound selected from the group consisting of:
(R)-[1,6]Naphthyridine-2-carboxylic acid (1-phenyl-ethyl)-amide,
(S)-[1,6]Naphthyridine-2-carboxylic acid (1-carbamoyl-2-phenyl-ethyl)-amide,
(S)-[1,6]Naphthyridine-2-carboxylic acid [1-carbamoyl-2-(4-hydroxy-phenyl)-ethyl]-amide,
[1,6]Naphthyridine-2-carboxylic acid ((R)-1-phenyl-ethyl)-amide,
[1,6]Naphthyridine-2-carboxylic acid 1-phenyl-propylamide,
(R)-[1,6]Naphthyridine-2-carboxylic acid 1-(naphth-2-yl)-ethylamide,
[1,6]Naphthyridine-2-carboxylic acid thiophen-2-yl-methylamide,
[1,6]Naphthyridine-2-carboxylic acid 1-(4-bromo-phenyl)-ethylamide,
[1,6]Naphthyridine-2-carboxylic acid 1-(4-chlorophenyl)-propylamide,
[1,6]Naphthyridine-2-carboxylic acid 1-(4-methylphenyl)-ethylamide,
[1,6]Naphthyridine-2-carboxylic acid 1-(4-chlorophenyl)-ethylamide,
[1,6]Naphthyridine-2-carboxylic acid 1-(4-methoxyphenyl)-ethylamide,
[1,6]Naphthyridine-2-carboxylic acid 1-(3-chlorophenyl)-propylamide,
[1,6]Naphthyridine-2-carboxylic acid 1-(4-fluorophenyl)-ethylamide,
[1,6]Naphthyridine-2-carboxylic acid 1-(4-methylphenyl)-propylamide,
[1,6]Naphthyridine-2-carboxylic acid 1-(thiophen-2-yl)-propylamide,
[1,6]Naphthyridine-2-carboxylic acid 1-(thiophen-3-yl)-ethylamide,
(R)-[1,6]Naphthyridine-2-carboxylic acid 1-(4-methylphenyl)-ethylamide,
(R)-[1,6]Naphthyridine-2-carboxylic acid 1-phenyl-propylamide, and
(R)-[1,6]Naphthyridine-2-carboxylic acid 1-(4-chloro-phenyl)-ethylamide.

8. Compound according to claim 1 for use as a pharmaceutically active agent.

9. Use of a compound having the general formula (I): 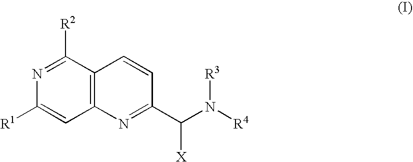
wherein
R¹, R², R²⁶-R⁶⁹ represent independently of each other —H, —OH, —OCH₃, —OC₂H₅, —OC₃H₇, —O-cyclo-C₃H₅, —OCH(CH₃)₂, —OC(CH₃)₃, —OC₄H₉, —OPh, —OCH₂-Ph, —OCPh₃, —SH, —SCH₃, —SC₂H₅, —SC₃H₇, —S-cyclo-C₃H₅, —SCH(CH₃)₂, —SC(CH₃)₃, —NO₂, —F, —Cl, —Br, —I, —N₃, —CN, —OCN, —NCO, —SCN, —NCS, —CHO, —COCH₃, —COC₂H₅, —COC₃H₇, —CO-cyclo-C₃H₅, —COCH(CH₃)₂, —COC(CH₃)₃, COOH, —COCN, —COOCH₃, —COOC₂H₅, —COOC₃H₇, —COO-cyclo-C₃H₅, —COOCH(CH₃)₂, —COOC(CH₃)₃, —OOC—CH₃, —OOC—C₂H₅, —OOC—C₃H₇, —OOC-cyclo-C₃H₅, —OOC—CH(CH₃)₂, —OOC—C(CH₃)₃, —CONH₂, —CONHCH₃, —CONHC₂H₅, —CONHC₃H₇, —CONH-cyclo-C₃H₅, —CONH[CH(CH₃)₂], —CONH[C(CH₃)₃], —CON(CH₃)₂, —CON(C₂H₅)₂, —CON(C₃H₇)₂, —CON(cyclo-C₃H₅)₂, —CON[CH(CH₃)₂]₂, —CON[C(CH₃)₃]₂, —NH₂, —NHCH₃, —NHC₂H₅, —NHC₃H₇, —NH-cyclo-C₃H₅, —NHCH(CH₃)₂, —NHC(CH₃)₃, —N(CH₃)₂, —N(C₂H₅)₂, —N(C₃H₇)₂, —N(cyclo-C₃H₅)₂, —N[CH(CH₃)₂]₂, —N[C(CH₃)₃]₂, —SOCH₃, —SOC₂H₅, —SOC₃H₇, —SO-cyclo-C₃H₅, —SOCH(CH₃)₂, —SOC(CH₃)₃, —SO₂CH₃, —SO₂C₂H₅, —SO₂C₃H₇, —SO₂-cyclo-C₃H₅, —SO₂CH(CH₃)₂, —SO₂C(CH₃)₃, —SO₃H, —SO₃CH₃, —SO₃C₂H₅, —SO₃C₃H₇, —SO₃-cyclo-C₃H₅, —SO₃CH(CH₃)₂, —SO₃C(CH₃)₃, —OCF₃, —OC₂F₅, —O—COOCH₃, —O—COOC₂H₅, —O—COOC₃H₇, —O—COO-cyclo-C₃H₅, —O—COOCH(CH₃)₂, —O—COOC(CH₃)₃, —NH—CO—NH₂, —NH—CO—NHCH₃, —NH—CO—NHC₂H₅, —NH—CO—NHC₃H₇, —NH—CO—NH-cyclo-C₃H₅, —NH—CO—NH[CH(CH₃)₂], —NH—CO—NH[C(CH₃)₃], —NH—CO—N(CH₃)₂, —NH—CO—N(C₂H₅)₂, —NH—CO—N(C₃H₇)₂, —NH—CO—N(cyclo-C₃H₅)₂, —NH—CO—N[CH(CH₃)₂]₂, —NH—CO—N[C(CH₃)₃]₂, —NH—CS—NH₂, —NH—S—NH-cyclo-C₃H₅, —NH—CS—NHC₃H₇, —NH—CS—NH[CH(CH₃)₂], —NH—CS—NH[C(CH₃)₃], —NH—CS—N(CH₃)₂, —NH—CS—N(C₂H₅)₂, —NH—CS—N(C₃H₇)₂, —NH—CS—N(cyclo-C₃H₅)₂, —NH—C(═NH)—NH₂, —NH—CS—NHC₂H₅, —NH—CS—NHCH₃, —O—CO—NH₂, —O—CO—NHCH₃, —O—CO—NHC₂H₅, —O—CO—NHC₃H₇, —O—CO—NH-cyclo-C₃H₅, —O—CO—NH[CH(CH₃)₂], —O—CO—NH[C(CH₃)₃], —O—CO—N(CH₃)₂, —O—CO—N(C₂H₅)₂, —O—CO—N(C₃H₇)₂, —O—CO—N(cyclo-C₃H₅)₂, —O—CO—N[CH(CH₃)₂]₂, —O—CO—N[C(CH₃)₃]₂, —O—COOCH₃, —OCO—C₂H₅, —O—CO—OC₃H₇, —O—CO-cyclo-C₃H₅, —O—CO—OCH(CH₃)₂, —O—CO—OC(CH₃)₃, —CH₂F —CHF₂, —CF₃, —CH₂Cl, —CHCl₂, —CCl₃, —CH₂Br —CHBr₂, —CBr₃, —CPh₃, —CH₂—CH₂F —CH₂—CHF₂, —CH₂—CF₃, —CH₂—CH₂Cl, —CH₂—CHCl₂, —CH₂—CCl₃, —CH₂—CH₂Br —CH₂—CHBr₂, —CH₂—CBr₃, —CH₃, —C₂H₅, —C₃H₇, -cyclo-C₃H₅, —CH(CH₃)₂, —C(CH₃)₃, —C₄H₉, —CH₂—CH(CH₃)₂, —CH(CH₃)—C₂H₅, —C(CH₃)₃, —C₅H₁₁, —C₆H₁₃, —C₇H₁₅, —C₈H₁₇, —C₉H₁₉, —C₁₀H₂₁, -Ph, —CH₂-Ph, —CH═CH₂, —CH₂—CH═CH₂, —C(CH₃)═CH₂, —CH═CH—CH₃, —C₂H₄—CH═CH₂, —CH═C(CH₃)₂, —C≡CH, —C═C≡CH₃, —CH₂—C≡CH;
R³, R¹¹ represent independently of each other —H, —CH₃, —C₂H₅, —C₃H₇, -cyclo-C₃H₅, —CH(CH₃)₂, —C(CH₃)₃, —C₄H₉, —CH₂—CH(CH₃)₂, —CH(CH₃)—C₂H₅, —C(CH₃)₃, —C₅H₁₁, —C₆H₁₃, —C₇H₁₅, —C₈H₁₇, —C₉H₁₉, —C₁₀H₂₁, -Ph, —CH₂-Ph, —CH═CH₂, —CH₂—CH═CH₂, —C(CH₃)═CH₂, —CH═CH—CH₃, —C₂H₄—CH═CH₂, —CH═C(CH₃)₂, —C≡CH, —C≡C—CH₃, —CH₂—C≡CH;
the moiety 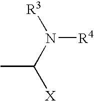
represents 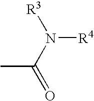
or R*;
R⁴ represents R** or 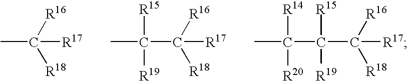
R* and R** represent independently of each other 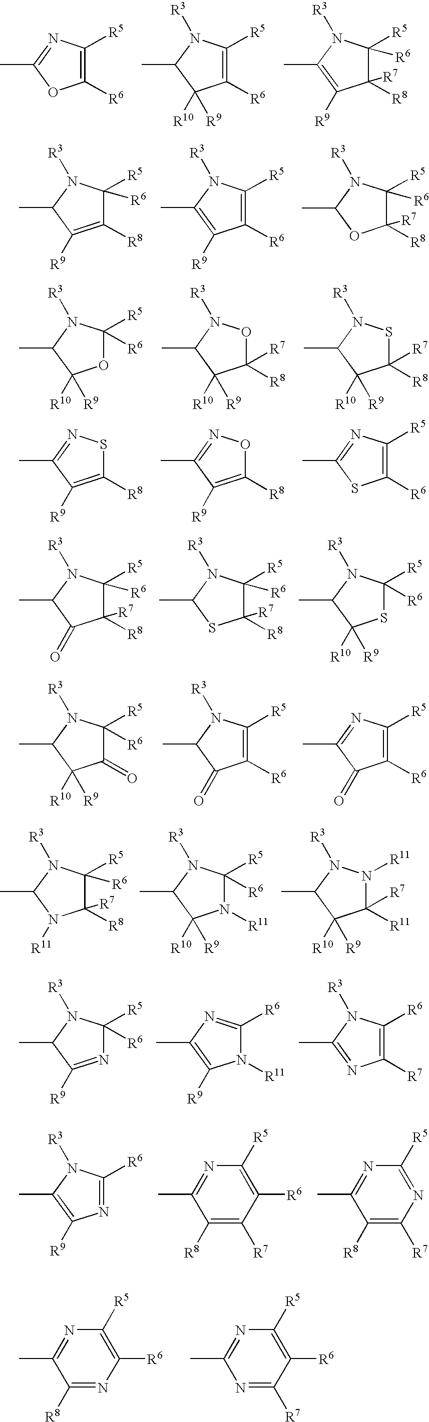 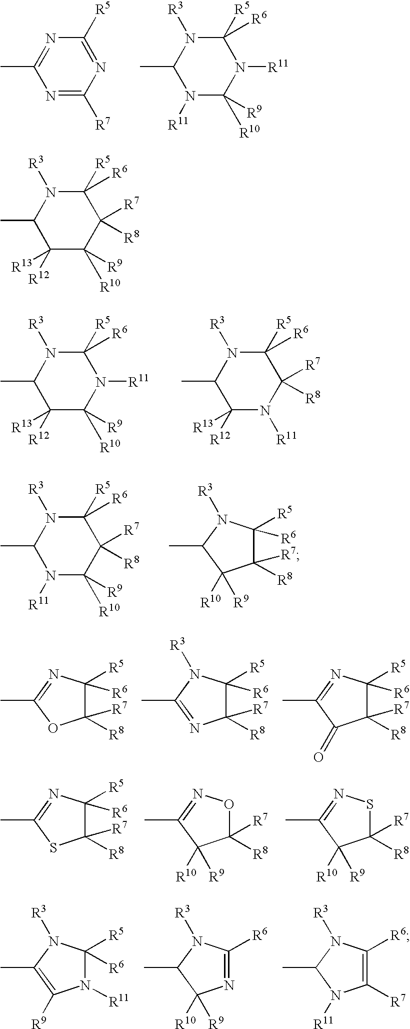
R# has the meanings as defined in claim 1;
R¹⁵, R¹⁷, R²⁰ represent independently of each other 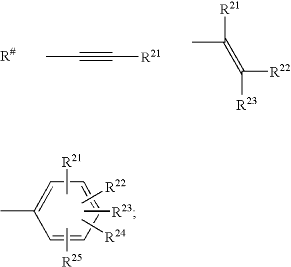
R⁵-R¹⁰, R¹²-R²⁵ represent independently of each other R⁵⁰-R⁶⁹, 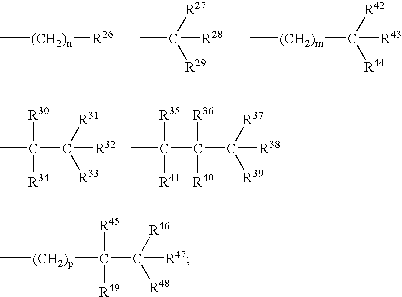
m, n, p are independently of each other integer from 0-10;
and stereoisomeric forms, prodrugs, solvates, hydrates and/or pharmaceutically acceptable salts thereof as inhibitor of the protein kinases ROCK1 and/or ROCK2.

10. Use of a compound according to claim 9 for prophylaxis and/or treatment of diseases associated with the enzyme ROCK2 or curable by inhibition of the enzymes ROCK1 and/or ROCK2.

11. Use of a compound according to claim 9 for prophylaxis and/or treatment of cancers, inflammation, infectious diseases, HIV, erectile dysfunction, cardiovascular diseases and disorders, hypertension, angina pectoris, cerebral ischaemia, cerebral vasospasm, myocardial ischaemia, coronary vasospasm, heart failure, myocardial hypertrophy, atherosclerosis, restenosis, spinal cord injuries, neuronal degeneration, thrombotic disorders, asthma, glaucoma, and osteoporosis.

12. Use of a compound according to claim 9 for the preparation of a medicament for the treatment of a disease selected from the list of cancers, inflammation, infectious diseases, HIV, erectile dysfunction, cardiovascular diseases and disorders, hypertension, angina pectoris, cerebral ischaemia, cerebral vasospasm, myocardial ischaemia, coronary vasospasm, heart failure, myocardial hypertrophy, atherosclerosis, restenosis, spinal cord injuries, neuronal degeneration, thrombotic disorders, asthma, glaucoma, and osteoporosis.

13. Use according to claim 12, wherein the cancer is selected from the group comprising:
adenocarcinoma, choroidal melanoma, acute leukemia, acoustic neurinoma, ampullary carcinoma, anal carcinoma, astrocytoma, basal cell carcinoma, pancreatic cancer, desmoid tumor, bladder cancer, bronchial carcinoma, breast cancer, Burkitt's lymphoma, corpus cancer, CUP-syndrome (carcinoma of unknown primary), colorectal cancer, small intestine cancer, small intestinal tumors, ovarian cancer, endometrial carcinoma, ependymoma, epithelial cancer types, Ewing's tumors, gastrointestinal tumors, gastric cancer, gallbladder cancer, gall bladder carcinomas, uterine cancer, cervical cancer, cervix, glioblastomas, gynecologic tumors, ear, nose and throat tumors, hematologic neoplasias, hairy cell leukemia, urethral cancer, skin cancer, skin testis cancer, brain tumors (gliomas), brain metastases, testicle cancer, hypophysis tumor, carcinoids, Kaposi's sarcoma, laryngeal cancer, germ cell tumor, bone cancer, colorectal carcinoma, head and neck tumors (tumors of the ear, nose and throat area), colon carcinoma, carniopharyngiomas, oral cancer (cancer in the mouth area and on lips), cancer of the central nervous system, liver cancer, liver metastases, leukemia, eyelid tumor, lung cancer, lymph node cancer (Hodgkin's/Non-Hodgkin's), lymphomas, stomach cancer, malignant melanoma, malignant neoplasia, malignant tumors gastrointestinal tract, breast carcinoma, rectal cancer, medulloblastomas, melanoma, meningiomas, Hodgkin's disease, mycosis fungoides, nasal cancer, neurinoma, neuroblastoma, kidney cancer, renal cell carcinomas, non-Hodgkin's lymphomas, oligodendroglioma, esophageal carcinoma, osteolytic carcinomas and osteoplastic carcinomas, osteosarcomas, ovarial carcinoma, pancreatic carcinoma, penile cancer, plasmocytoma, prostate cancer, pharyngeal cancer, rectal carcinoma, retinoblastoma, vaginal cancer, thyroid carcinoma, Schneeberger disease, esophageal cancer, spinalioms, T-cell lymphoma (mycosis fungoides), thymoma, tube carcinoma, eye tumors, urethral cancer, urologic tumors, urothelial carcinoma, vulva cancer, wart appearance, soft tissue tumors, soft tissue sarcoma, Wilm's tumor, cervical carcinoma and tongue cancer.

14. Use according to claim 12, wherein the infectious disease including opportunistic infection is selected from the group comprising: AIDS, Alveolar Hydatid Disease (AHD, Echinococcosis), Amebiasis (Entamoeba histolytica Infection), Angiostrongylus Infection, Anisakiasis, Anthrax, Babesiosis (Babesia Infection), Balantidium Infection (Balantidiasis), Baylisascaris Infection (Raccoon Roundworm), Bilharzia (Schistosomiasis), Blastocystis hominis Infection (Blastomycosis), Boreliosis, Botulism, Brainerd Diarrhea, Brucellosis, BSE (Bovine Spongiform Encephalopathy), Candidiasis, Capillariasis (Capillaria Infection), CFS (Chronic Fatigue Syndrome), Chagas Disease (American Trypanosomiasis), Chickenpox (Varicella-Zoster virus), Chlamydia pneumoniae Infection, Cholera, Chronic Fatigue Syndrome, CJD (Creutzfeldt-Jakob Disease), Clonorchiasis (Clonorchis Infection), CLM (Cutaneous Larva Migrans, Hookworm Infection), Coccidioidomycosis, Conjunctivitis, Coxsackievirus A16 (Hand, Foot and Mouth Disease), Cryptococcosis, Cryptosporidium Infection (Cryptosporidiosis), Culex mosquito (Vector of West Nile Virus), Cutaneous Larva Migrans (CLM), Cyclosporiasis (Cyclospora Infection), Cysticercosis (Neurocysticercosis), Dengue/Dengue Fever, Ebola Virus Hemorrhagic Fever, Echinococcosis (Alveolar Hydatid Disease), Encephalitis, Entomoeba coli Infection, Entomoeba dispar Infection, Entomoeba hartmanni Infection, Entomoeba histolytica Infection (Amebiasis), Entomoeba polecki Infection, Enterobiasis (Pinworm Infection), Enterovirus Infection (Non-Polio), Epstein-Barr Virus Infection, Escherichia coli Infection, Foodborne Infection, Foot and mouth Disease, Fungal Dermatitis, Gastroenteritis, Group A streptococcal Disease, Group B streptococcal Disease, Hansen's Disease (Leprosy), Hantavirus Pulmonary Syndrome, Helicobacter pylori Infection, Hematologic Disease, Hendra Virus Infection, Hepatitis (HCV, HBV), HIV Infection, Human Ehrlichiosis, Human Parainfluenza Virus Infection, Influenza, Isosporiasis (Isospora Infection), Lassa Fever, Leishmaniasis, Kala-azar (Kala-azar, Leishmania Infection), Leprosy, Lice (Body lice, Head lice, Pubic lice), Lyme Disease, Malaria, Marburg Hemorrhagic Fever, Measles, Meningitis, Mosquito-borne Diseases, Mycobacterium avium Complex (MAC) Infection, Naegleria Infection, Nosocomial Infections, Nonpathogenic Intestinal Amebae Infection, Onchocerciasis (River Blindness), Opisthorciasis (Opisthorcis Infection), Parvovirus Infection, Plague, PCP (Pneumocystis carinii Pneumonia), Polio, Q Fever, Rabies, Respiratory Syncytial Virus (RSV) Infection, Rheumatic Fever, Rift Valley Fever, River Blindness (Onchocerciasis), Rotavirus Infection, Roundworms Infection, Salmonellosis, Salmonella Enteritidis, Scabies, Shigellosis, Shingles, Sleeping Sickness, Smallpox, Streptococcal Infection, Tapeworm Infection (Taenia Infection), Tetanus, Toxic Shock Syndrome, Tuberculosis, Ulcers (Peptic Ulcer Disease), Valley Fever, Vibrio parahaemolyticus Infection, Vibrio vulnificus Infection, Viral Hemorrhagic Fever, Warts, Waterborne infectious Diseases, West Nile Virus Infection (West Nile Encephalitis), Whooping Cough, Yellow Fever, tuberculosis, leprosy, mycobacteria-induced meningitis, wherein Cytomegalovirus Infection is excluded.

15. Use of a compound according to claim 12, wherein the cardiovascular disease or disorder is selected from the group comprising:
adult congenital heart disease, aneurysm, stable angina, unstable angina, angina pectoris, angioneurotic edema, aortic valve stenosis, aortic aneurysm, arrhythmia, arrhythmogenic right ventricular dysplasia, arteriosclerosis, arteriovenous malformations, atrial fibrillation, Behcet syndrome, bradycardia, cardiac tamponade, cardiomegaly, congestive cardiomyopathy, hypertrophic cardiomyopathy, restrictive cardiomyopathy, cardiovascular disease prevention, carotid stenosis, cerebral hemorrhage, Churg-Strauss syndrome, diabetes, Ebstein's Anomaly, Eisenmenger complex, cholesterol embolism, bacterial endocarditis, fibromuscular dysplasia, congenital heart defects, heart diseases, congestive heart failure, heart valve diseases, heart attack, epidural hematoma, hematoma, subdural, Hippel-Lindau disease, hyperemia, hypertension, pulmonary hypertension, hypertrophic growth, left ventricular hypertrophy, right ventricular hypertrophy, hypoplastic left heart syndrome, hypotension, intermittent claudication, ischemic heart disease, Klippel-Trenaunay-Weber syndrome, lateral medullary syndrome, long QT syndrome mitral valve prolapse, moyamoya disease, mucocutaneous lymph node syndrome, myocardial infarction, myocardial ischemia, myocarditis, pericarditis, peripheral vascular diseases, phlebitis, polyarteritis nodosa, pulmonary atresia, Raynaud disease, restenosis, Sneddon syndrome, stenosis, superior vena cava syndrome, syndrome X, tachycardia, Takayasu's arteritis, hereditary hemorrhagic telangiectasia, telangiectasis, temporal arteritis, tetralogy of fallot, thromboanguitis obliterans, thrombosis, thromboembolism, tricuspid atresia, varicose veins, vascular diseases, vasculitis, vasospasm, ventricular fibrillation, Williams syndrome, peripheral vascular disease, varicose veins and leg ulcers, deep vein thrombosis, Wolff-Parkinson-White syndrome.

16. Use of a compound according to claim 1 in combination with an anti-HIV drug, antiproliferative drug, cytotoxic or cytostatic drug, ganciclovir, foscarnet, cidofovir, valganciclovir, fomivirsen, penciclovir or valaciclovir.

17. Pharmaceutical composition comprising at least one compound according to claim 1 as an active ingredient, together with at least one pharmaceutically acceptable carrier, excipient and/or diluents.
